(19) United States
(12) Patent Application Publication
(10) Pub. No.: US 2013/0338203 A1
(43) Pub. Date: Dec. 19, 2013

(54) TREATMENT OF ARTERIAL AGEING BY COMBINATION OF RAAS INHIBITOR AND HMG-CoA REDUCTASE INHIBITOR

(71) Applicant: Miso Sabovic, Ljubljana (SI)

(72) Inventor: Miso Sabovic, Ljubljana (SI)

(73) Assignee: FARMICOM PHARMACEUTICAL COMPANY D.O.O., Logatec (SI)

(21) Appl. No.: 14/001,816

(22) PCT Filed: Feb. 28, 2012
(86) PCT No.: PCT/EP2012/053360

§ 371 Date: Aug. 27, 2013

(30) Foreign Application Priority Data
Feb. 28, 2011 (SI) P-201100061
Dec. 9, 2011 (SI) P-201100458

(51) Int. Cl. A61K 31/4178; A61K 31/40; A61K 31/404; A61K 31/41; A61K 45/06

(52) U.S. Cl. CPC A61K 31/4178; A61K 31/41; A61K 45/06; A61K 31/404; A61K 31/40
USPC 514/381

(57) ABSTRACT

The present invention relates to a pharmaceutical combination composition comprising at least one renin-angiotensin-aldosterone system inhibitor in a subtherapeutic daily dose and at least one HMG-CoA reductase inhibitor in a subtherapeutic daily dose for use in prevention, reduction or reversal of arterial ageing in apparently healthy subjects.

### FIELD OF INVENTION

The present invention relates to a pharmaceutical composition comprising at least one renin-angiotensin-aldosterone system inhibitor in a subtherapeutic daily dose and at least one HMG-CoA reductase inhibitor in a subtherapeutic daily dose for use in the prevention, reduction or reversal of arterial aging in apparently healthy subjects. Further, the pharmaceutical composition according to the invention is also useful in decreasing the occurrence of cardiovascular disorders in apparently healthy subjects.

### BACKGROUND OF INVENTION

Ageing (British English) or aging (American English) is the accumulation of changes in an organism or object over time. Ageing in humans refers to a multidimensional process of physical, psychological and social change. Ageing is defined as the gradual biological impairment of normal function, probably as a result of changes made to cells, molecules and tissues/morphological components. These changes have a direct impact on the functional ability of organs such as for example the heart, brain, kidney and lungs, biological systems such as for example the nervous, digestive and reproductive system and ultimately the organism as a whole.

Although ageing affects the whole body the consequences of ageing are related to the involved organ or system. The ageing of arteries produces the most detrimental consequences of ageing. Ageing causes progressive decline in physiological arterial functions and morphology. Aged arteries generate changes in hemodynamic that importantly contribute to the development of cardiovascular diseases. In addition, aged arteries are more susceptible for the development of certain conditions such as atherosclerosis. Taken all facts together, arterial ageing substantially contributes to the development of cardiovascular diseases such as for example myocardial infarction, stroke, dementia, kidney failure, hypertension and similar. Thus, ageing, specifically arterial ageing, is one of most important risk factors for development of cardiovascular diseases. It is widely believed that ageing per se is not a modifiable risk factor. This conclusion does not necessarily apply to the arterial ageing, however. Cardiovascular diseases remain the leading cause of morbidity and mortality in developed countries despite current intensive management strategies. Importantly, up to date, no effective treatment that would be able to prevent, reduce or even reverse the process of arterial ageing has been disclosed.

Arterial ageing is characterized by alterations in cells, matrix, and biomolecules present in the arterial wall. Arterial ageing is a foundation for the initiation and progression of cardiovascular diseases. Although arterial ageing literary starts immediately after birth, it seems that important age-related changes occur at middle age. In this period (approximately between 20-65 years) age-related changes gradually and continuously progress. Basic representative functional and morphological age-related arterial changes are for example endothelial dysfunction, vascular smooth muscle cell proliferation/invasion/secretion, matrix fragmentation, collagenisation and glycation that result in typical age related changes such as for example increased arterial stiffness and decreased arterial wall elasticity. Age-associated arterial wall phenotype creates a microenvironment enriched with reactive oxygen species and inflammatory molecules. Several age-modified angiotensin II signaling molecules control and facilitate the processes producing age-related arterial changes. Age-related arterial changes are clinically silent, but as described above may lead to development of cardiovascular diseases. Targeting arterial ageing as soon as valuable can reduce the incidence/occurrence and progression of said cardiovascular diseases.

Arterial aging is a result of gradual changes of morphological (i.e. structural) and functional properties of the arterial wall. The arterial wall consists of three layers: intima, media and adventia. The most inner part of the arterial wall is endothelium (a part of intima), which is directly exposed to the blood in the artery lumen. There is a large amount of evidence providing that aging itself induces the stiffening of media and consequently the stiffening of whole arterial wall (morphological property) and the impairment of endothelial function (functional property).

It is well known in the art that arterial stiffness and endothelial dysfunction are among the most important mechanisms facilitating the development of cardiovascular disorders as hypertension, myocardial infarction, stroke, dementia, and similar. As regards the correlation between arterial aging, arterial stiffness, and cardiovascular risks, Mitchell at al. have found that increased arterial stiffness is a marker of increased cardiovascular risk, and arterial stiffness increases by aging. (Mitchell G F et al., Arterial stiffness and cardiovascular events: The Framingham Heart Study. Circulation 2010; 121:505-11). Thus, arterial aging, in particular affecting the gradual increase of arterial stiffness, increases the risk for cardiovascular disorders.

It is also well known in the art that one has to distinguish between arterial aging in apparently healthy subjects and arterial aging in connection with cardiovascular diseases (Najjar S. S. et al., Arterial Aging, Hypertension 2005; 46:454-462). When discussing apparently healthy subjects, Najjar et al. describe the changes in the arterial structure and function as part of “normative aging”, whereas when discussing cardiovascular diseases, they refer to accelerated changes which is not comparable to normative ageing. Furthermore, J. M. Bowness reports that changes in the composition of the extracellular matrix associated with normal aging are clearly different from those occurring in the development of advanced atherosclerotic lesions (J. M. Bowness, Atherosclerosis and aging of the arterial wall, Can Med Assoc J 1992; 147(2):201). Moreover, H.-Y.Lee et al. disclose that arterial walls stiffen with age and that this aging process in the arterial tree is heterogeneous, with distal arteries not exhibiting these stiffening changes, which is different from the atherosclerotic process (H.-Y. Lee et al., Circulation Journal 2010; 94; 2258-2262).

The renin-angiotensin-aldosterone system (RAAS) plays an important role in regulating blood volume and systemic vascular resistance, which together influence cardiac output and arterial pressure. As the name implies, there are three important components to this system: renin, angiotensin, and aldosterone. Renin, which is primarily released by the kidneys, stimulates the formation of angiotensin in blood and tissues, which in turn stimulates the release of aldosterone from the adrenal cortex. When renin is released into the blood, it acts upon a circulating substrate, angiotensinogen, that undergoes proteolytic cleavage to form the decapeptide angiotensin I. Vascular endothelium, particularly in the lungs, has an enzyme, angiotensin converting enzyme (ACE), that cleaves off two amino acids to form the octapeptide, angiotensin II (AII), although many other tissues in the body (heart, brain, vascular) also can form AII.

Renin inhibitors are antihypertensive drugs that inhibit the first and rate-limiting step of RAAS. Since the 1970s scientists have been trying to develop potent inhibitors with acceptable oral bioavailability. The first and second generations faced problems like poor bioavailability and lack of potency. The third generation is non-peptidic renin inhibitors with acceptable oral bioavailability and potency for clinical use in the treatment of hypertension.

Angiotensin-converting enzyme (ACE) inhibitors produce vasodilation by inhibiting the formation of angiotensin II. This vasoconstrictor is formed by the proteolytic action of renin (released by the kidneys) acting on circulating angiotensinogen to form angiotensin I. Angiotensin I is then converted to angiotensin II by angiotensin converting enzyme. ACE inhibitors also break down bradykinin (a vasodilator substance). Therefore, ACE inhibitors, by blocking the breakdown of bradykinin, increase bradykinin levels, which can contribute to the vasodilator action of ACE inhibitors. ACE inhibitors are used primarily to treat hypertension, they may also be prescribed for cardiac failure, diabetic nephropathy, renal disease, systemic sclerosis, left ventricular hypertrophy and other disorders. ACE inhibitors are often used in conjunction with a diuretic in treating hypertension and heart failure.

Angiotensin II receptor antagonists, also known as angiotensin receptor blockers (ARBs), AT1-receptor antagonists or sartans, are a group of pharmaceuticals which modulate the renin-angiotensin-aldosterone system. Their main use is in hypertension (high blood pressure), diabetic nephropathy (kidney damage due to diabetes), congestive heart failure, proteinuria, and prevention of cardiac remodeling after myocardial infarction. ARBs are receptor antagonists that block type 1 angiotensin II (AT₁) receptors on bloods vessels and also in other tissues as arterial wall and heart muscle. ARBs act on the surface and inside arterial wall.

HMG-CoA reductase inhibitors also known as statins are a class of drug used to lower cholesterol levels by inhibiting the enzyme HMG-CoA reductase that is the rate-controlling enzyme (EC 1.1.1.88) of the mevalonate pathway, the metabolic pathway that produces cholesterol and other isoprenoids. HMG-CoA reductase enzyme plays a central role in the production of cholesterol in the liver. Statins are among the most commonly prescribed drugs in medicine. Clinical studies have shown that statins significantly reduce the risk of heart attack and death in patients with proven coronary artery disease (CAD), and can also reduce cardiac events in patients with high cholesterol levels who are at increased risk for heart disease.

Anti-inflammatory refers to the property of a substance or treatment that reduces inflammation. Anti-inflammatory substances should suppress the expression induction of inflammatory functional proteins such as enzyme participating in the production of chemical mediator of various cytokines and inflammation, as well as suppress information transfer in cells participating in activation, and/or suppress the action expression by chemical mediator of various cytokines and inflammation.

An antioxidant is known as a molecule that can neutralize free radicals by accepting or donating an electron to eliminate the unpaired condition. Typically this means that the antioxidant molecule becomes a free radical in the process of neutralizing a free radical molecule to a non-free-radical molecule. But the antioxidant molecule will usually be a much less reactive free radical than the free radical neutralized. Therefore, an antioxidant inhibits the oxidation of other molecules. Oxidation is a chemical reaction that transfers electrons from a substance to an oxidizing agent. Oxidation reactions can produce free radicals. In turn, these radicals can start chain reactions that damage cells. Antioxidants terminate oxidation chain reactions by removing free radical intermediates, and inhibit other oxidation reactions.

Combinations of angiotensin II receptor antagonist and HMG-CoA reductase inhibitor and their therapeutically benefits for their primary indication are well known from the state of the art, for example numerous previous studies have shown that the combination of angiotensin II receptor antagonist and HMG-CoA reductase inhibitor in therapeutic doses are effective in treatment of hyperlipidemia and hypertension and cardiovascular disorders (e.g. Koh K K et al, Atherosclerosis 2010; 209: 307-13; Liu L et al, J Cardiovasc Pharmacol 2007; 50:50-5; Yoshikawa M et al, J Cardiovasc Pharmacol 2009; 53:179-86). The additional combinations with anti-inflammatory agents and/or antioxidants are known to be of certain treatment value for example for cardiovascular prevention (Antonpoulos A A et al. Recent Pat Cardiovasc Drug Disc 2009; 4:76-87). However, all the above mentioned references are silent on the effect on arterial ageing. Moreover, prior art does not teach or even does not give any hint that a subtherapeutic daily dose of these drugs is sufficient and efficient for prevention, reduction or reversal of arterial ageing in apparently healthy subjects.

WO 2005/072696 discloses the use of ACE (angiotensin-converting enzyme) inhibitors and/or angiotensin II receptor antagonists for the improving and/or maintaining the skin tone and for the treatment of skin ageing. The application is silent on the effect of said active substances on arterial ageing.

WO 2006/105806 discloses the composition comprising four or more active agents, namely a statin, a compound suppressing angiotensin production or activity and anti-inflammatory agent and at least one antioxidant used for prevention and/or treatment of ageing process. The application provides comparative data on a positive effect towards cell growth and cell reproduction when using the composition comprising all four above mentioned active agents. However, the application is silent on the impact of said combination on arterial ageing and it does not provide any data when one or more active agent is omitted from the composition.

Apparently novel approaches and strategies of prevention, reduction or reversal of arterial ageing are of great interest in view of the awareness that age, particularly arterial age is one of the most, if not, the most important risk factor for the development of cardiovascular disorders. Therefore, it would be a significant contribution to the art to provide an effective treatment of arterial ageing; this means treating functional and morphological changes of the arterial wall that are progressively developed during ageing per se.

It is known that angiotensin II receptor antagonists and HMG-CoA reductase inhibitors posses the so called pleiotropic effects, this means effects beyond their primary action. Pleiotropic effects of a drug are actions other than those for which the agent was specifically developed. These effects maybe related or unrelated to the primary mechanism of action of the drug, and they are usually unexpected.

It is an object of the present invention to provide a pharmaceutical composition which is suitable to prevent, reduce or reverse arterial ageing in apparently healthy subjects.

It is a further object of the present invention to provide a pharmaceutical composition which is suitable for improving the morphological properties of the arterial wall in apparently healthy subjects.

It is a further object of the present invention to improve the functional properties of the arterial wall such as the endothelial function in apparently healthy subjects.

It is a further object of the present invention to provide a composition which also provides a beneficial effect on the arterial aging after discontinuation of the treatment.

It is a further object of the present invention to provide a pharmaceutical composition which allows for a decrease in the occurrence of cardiovascular diseases.

### SUMMARY OF INVENTION

The objects of the present invention are surprisingly achieved by providing a pharmaceutical composition comprising at least one renin-angiotensin-aldosterone system inhibitor in a subtherapeutic daily dose and at least one HMG-CoA reductase inhibitor in a subtherapeutic daily dose, for use in the prevention, reduction or reversal of arterial ageing in apparently healthy subjects. More specifically, the present invention relates to the pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant and combinations thereof for use in the prevention, reduction or reversal of arterial ageing in apparently healthy subjects. An advantage of said pharmaceutical composition is a new approach for prevention of cardiovascular diseases. Accordingly, the pharmaceutical compositions according to the present invention are also useful in decreasing the occurrence of cardiovascular disorders in apparently healthy subjects.

In one aspect, the present invention is directed to a pharmaceutical composition comprising at least one renin-angiotensin-aldosterone system inhibitor in a subtherapeutic daily dose and at least one HMG-CoA reductase inhibitor in a subtherapeutic daily dose for use in the prevention, reduction or reversal of arterial ageing in apparently healthy subjects.

In a further aspect, the present invention is directed to a pharmaceutical composition comprising at least one renin-angiotensin-aldosterone system inhibitor in a subtherapeutic daily dose and at least one HMG-CoA reductase inhibitor in a subtherapeutic daily dose for use in decreasing the occurrence of cardiovascular disorders in apparently healthy subjects.

As used herein the term “arterial aging” refers to changes, in particular gradual changes of morphological (i.e. structural) and functional properties of the arterial wall. Preferably “arterial aging” exclusively refers to changes, in particular gradual changes of the morphological properties of the arterial wall.

As used herein, the morphological properties of the arterial wall are preferably to be understood as the stiffness properties of arteries. Preferably, arterial stiffness can be determined on the basis of the parameters pulse wave velocity (PWV) and β-stiffness.

Arterial stiffness is presently most adequately described by the parameter pulse wave velocity (PWV). The PWV is calculated from measurements of pulse transit time and the distance traveled by the pulse between two recording sites. Preferably, the PWV is measured on elastic arteries such as aorta, carotid artery, iliac artery, femoral artery. Thus, PWV represents the speed of pulse transmission through the arterial three. The stiffer the arteries are, the faster is the pulse transmission and consequently the higher is the PWV. The PWV can be easily and reproducibly measured using an ultrasound apparatus such as Aloka ProSound Alpha 10 apparatus with a high resolution eTracking system. Preferably the ultrasound apparatus is equipped with software for automatic determination of arterial stiffness parameters through the analysis of pulse waves. Other widely-used devices as Sphygmocor®, Compylor® and similar can be also used for PWV calculation.

The β-stiffness is also a parameter being a measure for arterial stiffness. It describes the local arterial stiffness. Accordingly, the determination of β-stiffness is a method for measuring stiffness from artery diameter and mutation width by the beating and blood pressure. Preferably β-stiffness is measured using a common carotid artery using an ultrasound apparatus such as Aloka ProSound Alpha 10 apparatus with a high resolution eTracking system. Preferably the ultrasound apparatus is equipped with software for automatic determination of arterial stiffness parameters through the analysis of pulse waves.

As used herein, the functional properties of the arterial wall are preferably characterized by the endothelial function of the arterial wall.

Endothelial function can be assessed with a variety of methods. The most widely used method is the ultrasound measurement of flow mediated dilatation (FMD) of brachial artery after short-term ischemia induced by sphygmomanometer inflation. Consequently, reactive hyperemia, which is dependent on endothelial function, occurs and brachial artery dilates. The present difference between the diameter measured after hyperemia and the basal diameter is taken as FMD. Generally FMD is used invasively with high-resolution ultrasound machines/systems; the measurements could be performed manually or automatically (as in the case when Aloka ProSound Alpha 10 apparatus is used).

In connection with “apparently healthy subjects” the term “arterial aging” preferably means that arterial aging in these subjects is not caused or accelerated by any extrinsic influence such as hypertension, metabolic syndrome, diabetes etc. In this regard, reference is made to FIG. 1 of the article by Lee et al., wherein the causes of arterial aging are presented (H.-Y. Lee et al., Circulation Journal 2010; 94; 2258-2262).

As used herein, “arterial aging” of “apparently healthy subjects” is preferably based on the structural change of the arteries with aging caused e.g. by longstanding arterial pulsation in the central artery, which has a direct effect on the structural matrix proteins, collagen and elastin in the arterial wall, disrupting muscular attachments and causing elastin fibers to fatigue and fracture. Further, accumulation of advanced glycation endproducts (AGE) on the proteins alters their physical properties and causes stiffness of the fibers in “apparently healthy subjects”. Still further, the calcium content in the arterial wall increases with age in “apparently healthy subjects”, which also contributes to arterial aging (H.-Y. Lee et al., Circulation Journal 2010; 94; 2258-2262).

As used herein, “apparently healthy subjects” are subjects, which have a low cardiovascular risk. An “apparently healthy subject” according to the present invention having a low cardiovascular risk exhibits a Framingham Risk Score for a coronary heart disease (CHD) (10-year risk) of 10% or less, preferably 8% or less, more preferably 5% or less. The Framingham Risk Score for the CHD is calculated on the basis described in “The Third Report of the National Cholesterol Education Program (NCEP) Expert Panel on Detection, Evaluation and Treatment of High Blood Cholesterol in Adults (Adult Treatment Panel III)”, Circulation 2002; 106: 3143-3421. The calculation of the Framingham Risk Score for a coronary heart disease (CHD) (10-year risk) is based on the ATP III page of the NHLBI Web site (www.nhlbi.nih.gov/guidelines/cholesterol) referenced at page 3229 of said article. The algorithm underlying the calculation of the Framingham risk equation in these calculators has been described by Anderson K M et al. in “An updated coronary risk profile. A statement for health professionals”, Circulation (1991), 83:356-362.

With the Framingham risk score for the CHD (10 years) the risk for coronary heart diseases such as myocardial infarction and death is assessed. For the present invention, an apparently healthy man has a Framingham Risk Score for the CHD of 10% or less, preferably 8% or less, more preferably 5% or less and an apparently healthy woman has a Framingham Risk Score for the CHD of 10% or less, preferably 8% or less, more preferably 5% or less.

The parameters included in the Framingham risk score for a CHD are as follows: gender, age, total cholesterol level, HDL cholesterol level, smoking, systolic blood pressure and untreated/treated hypertension.

An apparently healthy subject having a low cardiovascular risk does preferably not have a (manifested) cardiovascular disorder.

More preferably, the apparently healthy subject does not have diabetes.

In another preferred embodiment, the apparently healthy subject does not have a (manifested) cardiovascular disorder and in addition does not have disorders which importantly influence the functional capacity of different tissues/organs or the whole body.

The term cardiovascular disorder (CVD) according to the present invention refers to a cardiovascular disorder or cardiovascular event such as for example ischemic heart disease, carotid and intracerebral artery disease, peripheral arterial disease, aortic aneurism and the like, and any combinations thereof. Preferably CVD refers to myocardial infarction, stroke, dementia, critical limb ischemia, aortic aneurism and any combinations thereof, more preferably to myocardial infarction, stroke, vascular dementia and any combinations thereof.

As used herein, the term “subtherapeutic daily dose” in the context of the at least one HMG-CoA reductase inhibitor relates to a dose, which does not substantially change the cholesterol level. In this regard, the term “substantially” means that no therapeutic effect for the primary indication can be observed regarding these cholesterol levels. Preferably, the LDL cholesterol level is not lowered by more than 25%, preferably not more than 20%, more preferably not more than 18%. In another preferred embodiment, the HDL cholesterol level is not changed by more than 10%, preferably not more than 8%, more preferably not more than 5%. Preferably, the HDL cholesterol level is not decreased by more than 10%, preferably not more than 8%, more preferably not more than 5%.

The recommended daily therapeutic dose for a HMG-CoA reductase inhibitor is typically in the range of 10 mg to 80 mg. For example for the active Fluvastatin the recommended therapeutic daily dose is in the range of 40 mg to 80 mg. For atorvastatin the recommended therapeutic daily dose is in the range of 10 mg to 40 mg.

As used herein, the term “subtherapeutic daily dose” in the context of the at least one renin-angiotensin-aldosterone system inhibitor relates to a dose, which does not substantially lower the blood pressure. Preferably, the systolic blood pressure is not lowered by more than 15%, preferably not more than 10%. In another preferred embodiment, the diastolic blood pressure is not lowered by more than 15%, preferably not more than 10%.

The recommended daily therapeutic dose for an angiotension II receptor antagonist is usually in the range of 20 mg to 320 mg. For example for the active Valsartan the recommended therapeutic daily dose is in the range of 40 mg to 320 mg. For Losartan the recommended therapeutic daily dose is in the range of 25 mg to 100 mg.

The term “pharmaceutically acceptable salts” includes any and all non-toxic, salts of the disclosed compounds. Examples of pharmaceutically acceptable salts include inorganic and organic acid addition salts and basic salt. The pharmaceutically acceptable salts include, but are not limited to metal salts, such as sodium salt, potassium salt, cesium salt, and the like; alkaline earth metals, such as calcium salt, magnesium salt and the like, organic amine salts such as triethylamine salt, pyridine salt, picoline salt, ethanolamine salt, triethanolamine salt, dicyclohexylamine salt, N,N′-dibenzylethylenediamine salt and the like, inorganic acid salts, such as hydrochloride, hydrobromide, phosphate, sulphate and the like, organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like, and amino acid salts such as arginate, glutamate, and the like. Acid addition salts can be formed by mixing a solution of the particular compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts can be formed by mixing a solution of the particular compound of the present invention and a pharmaceutically acceptable non-toxic base such as sodium hydroxide, potassium hydroxide, choline hydroxide, sodium carbonate and the like.

The term “daily dose” of the pharmaceutically active ingredient(s) corresponds to the total amount of said active/the actives that is/are administered to a subject per day. The daily dose can be administered in any suitable frequency such as in a once-a-day dosage or alternatively in a divided dosage, e.g. twice-a-day dosage or dosages which have to be administered 3 or 4 times a day.

The term “residual improvement” refers to a change in the improvement of a parameter as measured after a certain time period (e.g. a rest period) in relation to the improvement achieved after a treatment period. The residual improvement after said time period is given as a percentage of the initial improvement (measured e.g. after determination of the treatment). As an example, the FMD at beginning of the treatment was 1%. The FMD measured after a treatment period was 4% (improvement 300%) and the FMD measured after a rest period following the treatment period was 3%, leading to a residual improvement of 67%.

The term “substantially”, if not defined otherwise in the context it is used, means that the value following the term may deviate ±10%, preferably ±5%.

The term “treatment period” as used herein is defined as the time period in which a subject is administered the daily dosis of the pharmaceutical composition according to the present invention.

The term “rest period” as used herein is defined as the time period in which a subject is not administered the pharmaceutical composition of the present invention.

### FIGURES

FIG. 1: Changes expressed in percentage of A) flow mediated dilation (FMD), B) β-stiffness of carotid artery and pulse wave velocity (PWV) in placebo and treated group after 1 month (30 days) of treatment (Example 1)

FIG. 2: Changes of plasma levels of hsCRP (% change in regard to initial concentration) after 1 month (30 days) treatment with placebo and treatment according to Example 1 (Example 2)

FIG. 3: Values of A) brachial artery flow mediated dilation (FMD); B) β-stiffness of common carotid artery and C) pulse wave velocity (PWV) at the beginning and at the end of the study in placebo and after 1 month (30 days) of treatment (Example 3)

FIG. 4: Changes expressed in percentage of A) flow mediated dilation (FMD) and B) pulse wave velocity (PWV) and β-stiffness of carotid artery in placebo and in after 1 month (30 days) treatment (Example 4)

FIG. 5: Changes expressed in percentage of A) flow mediated dilation (FMD) and B) pulse wave velocity (PWV) and β-stiffness of carotid artery in placebo and after 1 month (30 days) treatment (Example 5)

FIG. 6: Beneficial arterial characteristics (expressed in percentage of the effect achieved after 1 month (30 days) treatment) that still persist 5, 7 and 8 months after discontinuation of treatment according to Example 1 (Example 6)

FIG. 7: Beneficial arterial characteristics (expressed in percentage of the effect achieved after 1 month (30 days) treatment) that still persist 5, 7 and 10 months after discontinuation of treatment according to Example 3 (Example 7)

FIG. 8: Beneficial arterial characteristics (expressed in percentage of the effect achieved after 1 month (30 days) treatment) that still persist 7 months after discontinuation of treatment according to Example 1 or treatment with fluvastatin sodium or valsartan (Example 8)

FIG. 9: Changes of plasma levels of hsCRP (% change in regard to initial concentration) after 1 month (30 days) treatment with placebo, fluvastatin sodium, valsartan and treatment according to Example 1 (Example 9)

FIG. 10: Effect of the treatment according to Example 1 on “biological” arterial ageing (Example 10)

FIG. 11: Changes expressed in percentage of A) flow mediated dilation (FMD), B) β-stiffness of carotid artery and C) pulse wave velocity (PWV) in treated groups after 1 month (30 days) of treatment (Example 11)

FIG. 12: Improvements in pulse wave velocity (PWV) and β-stiffness after 1 month (30 days) treatment according to example 1 and influence on calculated arterial age by using age-related normograms (Example 1)

FIG. 13: Effects of cumulative addition of atorvastatin, losartan, and their combination on isolated rat aortic rings precontracted with 1 μM phenylephrine

FIG. 14: Effects of six weeks treatment with atorvastatin, losartan, and their combination on acetylcholine-induced endothelium-dependent vasorelaxation.

FIG. 15: Effects of six weeks treatment with atorvastatin, losartan, and their combination on the coronary flow values in experiments with 40-minute ischemia, followed by reperfusion.

FIG. 16: Changes expressed in percentage of flow mediated dilation (FMD), β-stiffness of carotid artery and pulse wave velocity (PWV) of treated group after 1 month (30 days) (1. intervention) and after a 2. Intervention (treatment for 1 month—30 days) after a 12-months rest period.

### DETAILED DESCRIPTION OF INVENTION

In one embodiment the present invention is directed to a pharmaceutical composition comprising at least one renin-angiotensin-aldosterone system inhibitor in a subtherapeutic daily dose, and at least one HMG-CoA reductase inhibitor in a subtherapeutic daily dose, for use in the prevention, reduction or reversal of arterial ageing in apparently healthy subjects.

In another embodiment the present invention is directed to a pharmaceutical composition comprising at least one renin-angiotensin-aldosterone system inhibitor in a subtherapeutic daily dose, and at least one HMG-CoA reductase inhibitor in a subtherapeutic daily dose, for use in decreasing the occurrence of cardiovascular disorders in apparently healthy subjects.

Without being bound to theory, the inventor believes that the prevention, reduction or reversal of arterial ageing, as evidenced for example by the reduction achieved by the pharmaceutical composition according to the present invention in the PWV and the β-stiffness will lead to a decrease in occurrence of cardiovascular disorders in apparently healthy subjects. The decrease in occurrence of the cardiovascular disorder may be indicated e.g. by the difference in the 10 year risk factor for CHD (Framingham Heart Study) calculated for the chronological age before the beginning of the treatment and the one calculated after the treatment using the calculated biological age.

The at least one renin-angiotensin-aldosterone inhibitor of the pharmaceutical composition of the present invention may be selected from the group consisting of renin inhibitor, angiotensin converting enzyme inhibitor, angiotensin II receptor antagonist and combinations thereof.

In a preferred embodiment of the present invention the at least one renin-angiotensin-aldosterone system inhibitor is an angiotensin II receptor antagonist.

Preferably the angiotensin II receptor antagonist is selected from the group consisting of azilsartan, losartan, eprosartan, irbesartan, olmesartan, candesartan, valsartan, telmisartan, and any pharmaceutically acceptable salts or esters and any combinations thereof. More preferably, the angiotensin II receptor antagonist is selected from the group consisting of azilsartan, losartan, olmesartan, candesartan, valsartan, telmisartan, and any pharmaceutically acceptable salts or esters, and combinations thereof, even more preferably the angiotensin II receptor antagonist is selected from the group consisting of azilsartan, losartan, candesartan, valsartan, telmisartan, and any pharmaceutically acceptable salts or esters, and combinations thereof, even more preferably the angiotensin II receptor antagonist is selected from the group consisting of losartan, valsartan, telmisartan, and any pharmaceutically acceptable salts or esters, and combinations thereof, or losartan, valsartan, and any pharmaceutically acceptable salts or esters, and combinations thereof.

In a particularly preferred embodiment of the present invention the angiotensin II receptor antagonist is valsartan or any pharmaceutically acceptable salt thereof, preferably valsartan.

In a further particularly preferred embodiment of the present invention the angiotensin II receptor antagonist is losartan or any pharmaceutically acceptable salt thereof, preferably losartan potassium.

In a preferred embodiment of the present invention the at least one HMG-CoA reductase inhibitor of the present invention is selected from the group consisting of mevastatin, pitavastatin, lovastatin, simvastatin, pravastatin, fluvastatin, atorvastatin, cerivastatin, rosuvastatin, and any pharmaceutically acceptable salts or esters, and combinations thereof. Preferably it is selected from the group consisting of simvastatin, fluvastatin, atorvastatin, rosuvastatin, and any pharmaceutically acceptable salts or esters, and combinations thereof, even more preferably it is selected from the group consisting of fluvastatin, atorvastatin, rosuvastatin, and any pharmaceutically acceptable salts or esters, and combinations thereof.

In a further preferred embodiment of the present invention the HMG-CoA reductase inhibitor is selected from the group consisting of fluvastatin, atorvastatin, and any pharmaceutically acceptable salts or esters, and combinations thereof.

In a particularly preferred embodiment of the present invention the at least one HMG-CoA reductase is fluvastatin or any pharmaceutically acceptable salt thereof, preferably fluvastatin sodium.

In a particularly preferred embodiment of the present invention the at least one HMG-CoA reductase is atorvastatin or any pharmaceutically acceptable salt thereof, preferably atorvastatin calcium. It is apparent for a person skilled in the art that the hemicalcium salt of atorvastatin is included in the term atorvatstatin calcium.

In one embodiment the pharmaceutical composition according to comprises compositions wherein the renin-angiotensin-aldosterone system inhibitor is selected from the group consisting of losartan, telmisartan, valsartan and any pharmaceutically acceptable salts or esters thereof, and the HMG-CoA reductase inhibitor is selected from the group consisting of fluvastatin and atorvastatin and any pharmaceutically acceptable salts or esters thereof.

In a further embodiment of the present invention the pharmaceutical composition comprises valsartan or a pharmaceutically acceptable salt or ester thereof and fluvastatin or a pharmaceutically acceptable salt or ester thereof. The use of valsartan and fluvastatin sodium is preferred.

In the pharmaceutical composition according to the present invention, the valsartan and fluvastatin sodium may be present in the composition in a weight ratio of from 9:1 to 1:9, preferably in a weight ratio of from 5:1 to 1:1, more preferably in a weight ratio of from 3:1 to 1:1, still more preferably in a weight ratio of from 2.5:1 to 1.6:1, most preferably in a weight ratio of 2:1.

In a further embodiment of the present invention the valsartan and fluvastatin sodium may be present in the composition in a weight ratio of from 9:1 to 1:9, preferably in a weight ratio of from 1:1 to 1:5, more preferably in a weight ratio of from 1:1 to 1:3, still more preferably in a weight ratio of from 1:1.6 to 1:2.5, most preferably in a weight ratio of 1:2.

In a further embodiment of the present invention, the renin-angiotensin-aldosterone system inhibitor comprises losartan or a pharmaceutically acceptable salt or ester thereof and the HMG-CoA reductase inhibitor comprises atorvastatin or a pharmaceutically acceptable salt or ester thereof, preferably the pharmaceutical composition may compriselosartan potassium and atorvastatin calcium.

The losartan potassium and atorvastatin calcium may be present in the composition in a weight ratio of from 9:1 to 1:9, preferably in a weight ratio of from 5:1 to 1:1, more preferably in a weight ratio of from 3.5:1 to 1.5:1, still more preferably in a weight ratio of from 3:1 to 2:1, most preferably in a weight ratio of 2.5:1.

In another embodiment the losartan potassium and atorvastatin calcium may be present in the composition in a weight ratio of from 20:1 to 1:1, preferably in a weight ratio of from 12:1 to 5:1, more preferably in a weight ratio of from 10:1 to 6:1, still more preferably in a weight ratio of from 9:1 to 7:1, most preferably in a weight ratio of 8:1.

In one embodiment of the invention the subtherapeutic daily dose of the renin-angiotensin-aldosterone system inhibitor and the subtherapeutic daily dose of the HMG-CoA reductase inhibitor correspond to doses which together do not substantially change the cholesterol levels. In a preferred embodiment the LDL cholesterol levels are nor lowered by more than 25%, preferably more than 20%, more preferably more than 18%, when administered for at least 10 days, preferably at least 14 days, more preferably at least 1 month. In yet another embodiment of the invention the subtherapeutic daily dose of the renin-angiotensin-aldosterone system inhibitor and the subtherapeutic daily dose of the HMG-CoA reductase inhibitor correspond to doses which together do not change or decrease the HDL cholesterol levels by more than 10%, preferably more than 8%, more preferably more than 5%, when administered for at least 10 days, preferably at least 14 days, more preferably at least 1 month.

In one embodiment of the present invention, the subtherapeutic daily dose of the renin-angiotensin-aldosterone system inhibitor and the subtherapeutic daily dose of the HMG-CoA reductase inhibitor correspond to doses which together do not lower the systolic blood pressure in a subject by more than 15%, preferably more than 10%, more preferably more than 6%, most preferably more than 4%, when administered for at least 10 days, preferably at least 14 days, more preferably at least 1 month.

In yet another embodiment of the present invention, the subtherapeutic daily dose of the renin-angiotensin-aldosterone system inhibitor and the subtherapeutic daily dose of the HMG-CoA reductase inhibitor correspond to doses which together do not lower the diastolic blood pressure in a subject by more than 15%, preferably more than 10%, more preferably more than 8%, most preferably more than 5%, when administered for at least 10 days, preferably at least 14 days, more preferably at least 1 month.

In yet another embodiment of the present invention the subtherapeutic daily dose of the renin-angiotensin-aldosterone inhibitor is between 1 and 50%, preferably between 1 and 25% of the daily recommended therapeutic dose for the primary medical indication of said active.

The subtherapeutic daily dose of the renin-angiotensin-aldosterone inhibitor is preferably between 1 and 75 mg, between 1 and 60 mg, between 1 and 50 mg, between 1 and 45 mg, between 1 and 40 mg, and/or between 1 and 25 mg. In another embodiment the daily dose of the renin-angiotensin-aldosterone inhibitor is selected from 10, 15, 20, 25, or 30 mg.

In one embodiment of the present invention the renin-angiotensin-aldosterone is valsartan or any pharmaceutically acceptable salts or esters thereof, and the subtherapeutic daily dose is between 1 and 75 mg, preferably between 1 and 60 mg, more preferably between 1 and 50 mg, still more preferably between 1 to 40 mg, most preferably between 10 to 30 mg, particularly preferably 20 mg.

In one embodiment of the present invention the renin-angiotensin-aldosterone is telmisartan or any pharmaceutically acceptable salts or esters thereof, and the subtherapeutical dose thereof is between 1 to 40 mg, preferably between 1 to 20 mg, most preferably 20 mg.

In one embodiment of the present invention the renin-angiotensin-aldosterone is losartan or any pharmaceutically acceptable salts or esters thereof, and the subtherapeutical dose thereof is between 1 to 40 mg, preferably between 1 to 30 mg, more preferably 20 mg and most preferably 12.5 mg.

In one embodiment of the present invention the subtherapeutic daily dose of the HMG-CoA reductase inhibitor is between 1 to 35 mg, preferably between 1 to 30 mg, more preferably between 1 and 25 mg, still more preferably between 1 and 20 mg, most preferably between 1 and 15 mg, particularly preferably between 1 and 12 mg. In another embodiment the daily dose of the HMG-CoA reductase inhibitor is selected from 5, 10, 15, 20, or 25 mg.

In one embodiment of the present invention the HMG-CoA reductase inhibitor is fluvastatin or any pharmaceutically acceptable salts or esters thereof, and the subtherapeutical dose thereof is between 1 to 20 mg, preferably between 1 to 10 mg, most preferably 10 mg.

In one embodiment of the present invention the HMG-CoA reductase inhibitor is atorvastatin or any pharmaceutically acceptable salts or esters thereof, and the subtherapeutical dose thereof is between 1 to 10 mg, preferably between 1 to 5 mg, most preferably 5 mg.

The present invention is directed to a pharmaceutical composition comprising at least one renin-angiotensin-aldosterone system inhibitor in a subtherapeutic daily dose and at least one HMG-CoA reductase inhibitor in a subtherapeutic daily dose for use in the prevention, reduction or reversal of arterial aging in apparently healthy subjects.

The “apparently healthy subjects” have a low cardiovascular risk.

An apparently healthy subject having a low cardiovascular risk exhibits a Framingham Risk Score for a CHD (10-year risk) of 10% or less, preferably 8% or less, more preferably 5% or less as defined above.

Preferably the apparently healthy subject does not have a (manifested) cardiovascular disorder. Preferably, the apparently healthy subject does not have a (manifested) cardiovascular disorder and in addition does not have disorders which importantly influence the functional capacity of different tissues/organs or the whole body.

The term cardiovascular disorder (CVD) according to the present invention refers to a cardiovascular disorder or cardiovascular event such as for example ischemic heart disease, carotid and intracerebral artery disease, peripheral arterial disease, aortic aneurism and the like, and any combinations thereof. Preferably CVD refers to myocardial infarction, stroke, dementia, critical limb ischemia, aortic aneurism and any combinations thereof, more preferably to myocardial infarction, stroke, vascular dementia and any combinations thereof.

In one embodiment, the apparently healthy subject is a mammal, preferably a human subject.

In one aspect of the present invention, the pharmaceutical composition comprising at least one renin-angiotensin-aldosterone system inhibitor in a subtherapeutic daily dose and at least one HMG-CoA reductase inhibitor in a subtherapeutic daily dose is useful in the prevention, reduction or reversal of arterial aging is achieved after treatment for at least one week, at least two weeks, preferably between 2 weeks and 3 months, more preferably between 2 weeks and 2 months, and more preferably between 2 weeks and 1 month, and most preferably after treatment for 1 month (e.g. 30 or 31 days).

In one embodiment the flow-mediated dilatation of brachial artery (FMD) after a period of treatment, preferably after 1 month of treatment, compared to the beginning of the treatment is increased. In a preferred aspect of this embodiment, the FMD increases by at least 50%, preferably at least 75%, more preferably at least 100%, more preferably at least 150%, still more preferably at least 200%, most preferably at least 250%, particularly preferably 275% after 1 month of treatment compared to the beginning of the treatment.

In another embodiment of the present invention, a decrease of the pulse-wave velocity (PWV) after a period of treatment, preferably after 1 month of treatment, compared to the beginning of the treatment is achieved. In a preferred aspect of this embodiment the PWV decreases by at least 3%, preferably at least 5%, more preferably at least 8%, most preferably at least 10% after 1 month of treatment compared to the beginning of the treatment.

In yet another embodiment of the present invention, the β-stiffness of carotid artery after a period of treatment, preferably after 1 month of treatment, compared to the beginning of the treatment is decreased. In a preferred aspect of this embodiment the β-stiffness decreases by at least 5%, preferably at least 8%, more preferably at least 12%, and most preferably at least 16% after 1 month of treatment compared to the beginning of the treatment.

The effect on arterial aging may be determined by measuring the difference in the parameters of the pulse-wave velocity (PWV) and the β-stiffness of carotid artery after 1 month of treatment compared to the beginning of the treatment.

The effect achieved by a pharmaceutically active substance is usually determined by the presence of a therapeutically effective concentration of said active in the blood. Therefore, the half-life time (t_{1/2}) in the blood plasma is an important factor which influences the period of time for which efficacy of a dosis regimen may be observed. For example the half-life time for fluvastatin is about 2.5 hours, for losartan it about 1.5-2 hours, for atorvastatin about 12-14 hours and for valsartan about 6.0 hours, respectively, Therefore, it can be expected that a pharmaceutical effect can only be maintained as long as the pharmaceutically active substance is administered on a regular basis, e.g. on a daily basis, twice-a-days-basis, or the like. This is also reflected by the treatment schedule of the primary indications of the renin-angiotensin-aldosterone system inhibitors or the HMG-CoA reductase inhibitors.

It has surprisingly been found by the inventor of the present invention that the use of the pharmaceutical composition according to the present invention comprising at least one renin-angiotensin-aldosterone system inhibitor in a subtherapeutic daily dose and at least one HMG-CoA reductase inhibitor in a subtherapeutic daily dose allows for an exceptionally long term persistence of improvements achieved for the arterial characteristics after discontinuation of the treatment resulting in long term improvement of arterial wall properties such as the arterial ageing. This improvement is reflected for example by the residual of the improvement after a certain “rest period” compared to the improvement after the treatment period. The phenomenon is described in the present invention by the term “rest period”. In the art also the term “activity during drug-free period” is used. The two terms “rest period” and “activity during drug-free period” are used interchangeably.

In one embodiment of the present invention the reduction or reversal of arterial aging after a period of treatment persists in a substantial amount the for at least 1 month, preferably at least 3 months, more preferably at least 5 months, still more preferably at least 7 months, most preferably at least 10 months, particularly preferably for approximately at least 12 months after discontinuation of the treatment.

In one aspect of this embodiment, after discontinuation of the treatment for at least 1 month the residual improvement of the PWV is at least 40%, preferably at least 50%, more preferably at least 65%, most preferably at least 70% based on the decrease of the PWV after a period of treatment. In another aspect of this embodiment, after discontinuation of the treatment for at least 3 months the residual improvement of the PWV is at least 40%, preferably at least 50%, more preferably at least 65%, most preferably at least 70% based on the decrease of the PWV after a period of treatment. In one aspect of this embodiment, after discontinuation of the treatment for at least 5 months the residual improvement of the PWV is at least 40%, preferably at least 50%, more preferably at least 65%, most preferably at least 70% based on the decrease of the PWV after a period of treatment. In a preferred aspect of this embodiment after discontinuation of the treatment for at least 5 months the residual improvement of the PWV is at least 40%, preferably at least 50%, more preferably at least 65%, most preferably at least 70% based on the decrease of the PWV after 1 month of treatment. In another preferred aspect of this embodiment after discontinuation of the treatment for at least 7 months the residual improvement of the PWV is at least 35%, preferably at least 45%, more preferably at least 60%, most preferably at least 65% based on the decrease of the PWV after a treatment period, preferably 1 month of treatment.

In a preferred aspect of this embodiment after discontinuation of the treatment for at least 8 months the residual improvement of the PWV is at least 5%, preferably at least 10%, more preferably at least 20%, most preferably at least 25% based on the decrease of the PWV after a treatment period, preferably 1 month of treatment.

In another aspect of the invention, after discontinuation of the treatment for at least 1 month the residual improvement of the β-stiffness is at least 40%, preferably at least 50%, more preferably at least 65%, and most preferably at least 70% based on the decrease of the β-stiffness after a period of treatment. In another aspect of this embodiment, after discontinuation of the treatment for at least 3 months the residual improvement of the β-stiffness is at least 40%, preferably at least 50%, more preferably at least 65%, and most preferably at least 70% based on the decrease of the β-stiffness after a period of treatment. In another aspect of the invention, after discontinuation of the treatment for at least 5 months the residual improvement of the β-stiffness is at least 40%, preferably at least 50%, more preferably at least 65%, and most preferably at least 70% based on the decrease of the β-stiffness after a period of treatment.

In a preferred aspect of this embodiment after discontinuation of the treatment for at least 5 months the residual improvement of the β-stiffness is at least 40%, preferably at least 50%, more preferably at least 65%, and most preferably at least 70% based on the decrease of the β-stiffness after 1 month of treatment. In another preferred aspect of this embodiment after discontinuation of the treatment for at least 7 months the residual improvement of the β-stiffness is at least 35%, preferably at least 45%, more preferably at least 60%, and most preferably at least 65% based on the decrease of the β-stiffness after 1 month of treatment. In a preferred aspect of this embodiment after discontinuation of the treatment for at least 8 months the residual improvement of the β-stiffness is at least 5%, preferably at least 10%, more preferably at least 20% based on the decrease of the β-stiffness after 1 month of treatment. In a preferred aspect of this embodiment the β-stiffness is still decreased by more than 60%, preferably more than 65%, more preferably by 68% after 1 month of treatment and after 7 months discontinuation of the treatment.

In another embodiment of the present invention, the improvement on the endothelial function persists in a substantial amount for at least 1 month, preferably at least 3 months, more preferably at least 5 months, still more preferably at least 7 months, most preferably at least 10 months, particularly preferably for approximately at least 12 months after discontinuation of the treatment. In one aspect of this embodiment, after discontinuation of the treatment for at least 1 months the residual improvement of the FMD is at least 50%, preferably at least 60%, more preferably at least 75%, most preferably at least 85% based on the increase of the FMD after a period of treatment. In one aspect of this embodiment, after discontinuation of the treatment for at least 3 months the residual improvement of the FMD is at least 50%, preferably at least 60%, more preferably at least 75%, most preferably at least 85% based on the increase of the FMD after a period of treatment. In one aspect of this embodiment, after discontinuation of the treatment for at least 5 months the residual improvement of the FMD is at least 50%, preferably at least 60%, more preferably at least 75%, most preferably at least 85% based on the increase of the FMD after a period of treatment. In a preferred aspect of this embodiment after discontinuation of the treatment for at least 5 months the residual improvement of the FMD is at least 50%, preferably at least 60%, more preferably at least 75%, most preferably at least 85% based on the increase of the FMD after 1 month of treatment. In a preferred aspect of this embodiment the FMD is still increased by more than 70%, preferably by more than 80% after 1 month of treatment and after 7 months discontinuation of the treatment. In another preferred aspect of this embodiment after discontinuation of the treatment for at least 7 months the residual improvement of the FMD is at least 40%, preferably at least 50%, more preferably at least 65%, more preferably at least 75%, most preferably at least 80% based on the increase of the FMD after 1 month of treatment. In a preferred aspect of this embodiment after discontinuation of the treatment for at least 8 months the residual improvement of the FMD is at least 20%, preferably at least 30%, more preferably at least 40%, most preferably at least 50% based on the increase of the FMD after 1 month of treatment.

In one embodiment of the present invention the plasma concentration of high sensitivity C-reactive protein (hsCRP) is decreased by at least 5%, preferably at least 10%, more preferably at least 15%, most preferably approximately 20% after a period of treatment. In a preferred embodiment of the invention, the plasma concentration of high sensitivity C-reactive protein (hsCRP) is decreased by at least 5%, preferably at least 10%, more preferably at least 15%, most preferably approximately 20% after 1 month of treatment.

In one embodiment of the present invention, the pharmaceutical composition according to the present invention for use in the prevention, reduction or reversal of arterial aging is applied in a repeated intervention cycle comprising at least one treatment-period followed by at least one rest-period. The intervention cycle is preferably repeated at least once, more preferably 2, 3, 4 or 5 times.

The treatment-period may last at least one week, at least two weeks, preferably between 2 weeks to 3 months, more preferably between 2 weeks and 2 months, still more preferably between 2 weeks and 1 month (e.g. 30 or 31 days).

The rest-period may be at least 1 day, preferably at least 1 week, more preferably at least 1 month, more preferably at least 3 months, still more preferably at least 4 months, more preferably at least 6 months, most preferably at least 8 months, particularly preferably approximately 10 or 12 months.

In a preferred embodiment of the invention, a first treatment period is 1 month, followed by a 12 months rest period, again followed by a second treatment period of 1 month.

The pharmaceutical composition according the present invention comprising at least one renin-angiotensin-aldosterone system inhibitor in a subtherapeutic daily dose and at least one HMG-CoA reductase inhibitor in a subtherapeutic daily dose may comprise one or more further active agents, preferably selected from the group consisting of an anti-inflammatory agent, an antioxidant, and combinations thereof.

The anti-inflammatory agents and/or the antioxidants may be selected from the group defined below.

In one embodiment of the present invention, the pharmaceutical composition may further comprise an anti-inflammatory agent and an antioxidant, which is not vitamin C or vitamin E.

In another embodiment of the present invention, the pharmaceutical composition may further comprise an anti-inflammatory agent, but not comprising an antioxidant.

In yet another embodiment of the present invention the pharmaceutical composition may comprise an antioxidant and an anti-inflammatory agent, wherein the anti-inflammatory agent is not acetylsalicylic acid.

In yet another embodiment of the present invention, the pharmaceutical composition may further comprise an antioxidant, but not comprising an anti-inflammatory agent.

In yet another embodiment of the present invention, the pharmaceutical composition may further comprise an anti-inflammatory agent and/or an antioxidant. In a preferred embodiment the anti-inflammatory agent is selected from the group consisting of acetylsalicylic acid and resveratrol. In another preferred embodiment the antioxidant is coenzyme Q10 or any analogue thereof. In a preferred embodiment of the present invention the anti-inflammatory agent is acetylsalicylic and the antioxidant is coenzyme Q10.

In case acetylsalicylic acid is present in the pharmaceutical composition according to the present invention it is present in an amount, which corresponds to a weight ratio of acetylsalicylic acid and HMG-CoA reductase inhibitor of from 30:1 to 1:1, preferably 20:1 to 5:1, more preferably 12:1 to 8:1, most preferably 10:1. In another embodiment of the present invention, the pharmaceutical composition comprises acetylsalicylic acid in a daily dose of between 1 to 200 mg, preferably 50 to 150 mg, most preferably 100 mg.

In case coenzyme Q10 is present in the pharmaceutical composition according to the present invention it is present in an amount which corresponds to a weight ratio of acetylsalicylic acid and HMG-CoA reductase inhibitor of from 30:1 to 1:1, preferably 20:1 to 5:1, more preferably 12:1 to 8:1, most preferably 10:1. In another embodiment of the present invention the pharmaceutical composition comprises coenzyme Q10 in a daily dose of 1 to 200 mg, preferably 50 to 150 mg, most preferably 100 mg.

In a preferred embodiment of the present invention the pharmaceutical composition comprises valsartan or any pharmaceutically acceptable salts thereof in a daily dose between 1 and 40 mg, preferably between 10 to 30 mg, most preferably 20 mg, and fluvastatin or any pharmaceutically acceptable salts thereof in a daily dose between 1 and 20 mg, preferably between 1 and 10 mg, most preferably 10 mg, and acetylsalicylic acid, resveratrol and/or coenzyme Q10 in any pharmaceutically acceptable form.

In a preferred embodiment of the present invention the pharmaceutical composition comprises valsartan or any pharmaceutically acceptable salts thereof in a daily dose between 1 and 40 mg, preferably between 10 to 30 mg, most preferably 20 mg, and fluvastatin or any pharmaceutically acceptable salts thereof in a daily dose between 1 and 20 mg, preferably between 1 and 10 mg, most preferably 10 mg, and acetylsalicylic acid in a daily dose between 1 to 200 mg, preferably 50 to 150 mg, most preferably 100 mg and/or coenzyme Q10 1 to 200 mg, preferably 50 to 150 mg, most preferably 100 mg.

In one embodiment, the present invention relates to a pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times. One intervention-cycle consists of one treatment-period lasting between about 2 weeks to about 3 months, preferably between about 2 weeks to about 2 months and more preferably between about 2 weeks to about 1 month and one rest-period lasting approximately 12 months, preferably between 6 and 12 months.

In another embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and optionally acetylsalicylic acid, resveratrol and/or coenzyme Q10 in any pharmaceutically acceptable form.

In another embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and optionally acetylsalicylic acid, resveratrol and/or coenzyme Q10 in any pharmaceutically acceptable form for use in prevention, reduction or reversal of arterial ageing in apparently healthy subjects.

In another embodiment, the present invention is the pharmaceutical composition comprising valsartan or any pharmaceutically acceptable salts thereof in a subtherapeutic daily dose and fluvastatin or any pharmaceutically acceptable salts thereof in a subtherapeutic daily dose and optionally acetylsalicylic acid, resveratrol and/or coenzyme Q10 in any pharmaceutically acceptable form for use in decreasing occurrence of cardiovascular disorders in apparently healthy subjects.

Further embodiments of the invention relate to the pharmaceutical composition according to present invention further comprising one or more pharmaceutically acceptable excipient.

One of the embodiment of the present invention is the pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

The inventors of the present application surprisingly found out that the pharmaceutical composition according to the present invention provides synergistic effect in comparison to their individual constituents, these are at least one renin-angiotensin-aldosterone system inhibitor and at least one HMG-CoA reductase inhibitor (the efficacy of angiotensin II receptor antagonist alone and the efficacy of HMG-CoA reductase inhibitor alone towards arterial ageing are described in a co-pending patent applications), by substantially improving both functional characteristics such as for example endothelial function measured by flow-mediated dilatation of brachial artery (FMD) and morphological characteristics such as for example stiffness and elasticity of arteries measured by pulse-wave velocity (PWV) and β-stiffness of carotid artery. It is important to emphasize that there is a similarity between the terms morphological or structural which are used to describe the same characteristics by different authors but basically disclose the same characteristics of arterial wall. All three above mentioned methods FMD, PWV and β-stiffness are standard in the art and generally widely accepted methods for estimation of functional or morphological characteristics of arteries. When administering the pharmaceutical composition according to the present invention the significant and unexpected improvement in all above mentioned characteristics are observed. Furthermore, by using age-related normogram (according to the methods described in Jurasic M J et al. β-stiffness—setting age standards. Acta Clin Croat 2009; 48:253-8; and Carerj S. Normal vascular ageing evaluated by a new tool: e-tracking. Eur J Echocardiography 2006; suppl 1:S49.) obtained on large sample of apparently healthy subjects—who have a low cardiovascular risk as defined above—the estimation of “biological” arterial age can be determined. The inventors of the present application surprisingly found out that there is a significant and unexpected decrease of “biological” arterial age by applying the pharmaceutical composition of the present invention after treatment.

The term renin-angiotensin-aldosterone system (RAAS) inhibitor as used in the present invention can include renin inhibitor, angiotensin-converting enzyme (ACE) inhibitor and angiotensin II receptor antagonist and any combinations thereof.

The renin inhibitor can be aliskiren.

The term ACE inhibitor as used in the present invention can include, but is not limited to, benazepril, captopril, enalapril, fosinopril, lisinopril, perindopril, trandolapril, moexipril, quinapril, ramipril and any pharmaceutically acceptable salts or esters thereof. Preferably ACE inhibitor can be selected from the group consisting of, but not limited to, perindopril, lisinopril, enalapril, moexipril, ramipril and any pharmaceutically acceptable salts or esters thereof, more preferably it can be selected from the group consisting of, but not limited to, perindopril and ramipril and any pharmaceutically acceptable salts or esters thereof.

The term angiotensin II receptor antagonist as used in the present invention can include, but is not limited to, azilsartan, losartan, eprosartan, irbesartan, olmesartan, candesartan, valsartan and telmisartan and any pharmaceutically acceptable salts or esters thereof. Preferably angiotensin II receptor antagonist can be selected from the group consisting of, but not limited to, azilsartan, losartan, telmisartan, olmesartan, candesartan and valsartan and any pharmaceutically acceptable salts or esters thereof, more preferably it can be selected from the group consisting of, but not limited to, losartan, telmisartan, azilsartan, candesartan and valsartan and any pharmaceutically acceptable salts or esters thereof and even more preferably angiotensin II receptor antagonist is valsartan and telmisartan and any pharmaceutically acceptable salts or esters thereof.

Moreover, the term RAAS inhibitor as used in the present invention can further include one or more combination with other active substance such as for example, but not limited to, combination with diuretic, such as for example thiazide such as for example chlorothiazide, chlorthalidone, hydrochlorothiazide, hydroflumethiazide, indapamide, methyclothiazide, metolazone, polythiazide, preferably hydrochlorothiazide and indapamide; loop diuretic such as for example bumetanide, ethacrynic acid, furosemide, torsemide, preferably furosemide and torsemide; K+-sparing diuretic such as for example amioloride, eplerenone, spironolactone, triamterene, preferably eplerenone and spironolactone; and Ca-inhibitors such as for example acetazolamide, dichlorphenamide, methazolamide; more preferably hydrochlorothiazide; combination with calcium channel blockers such as for example dihydropiridine calcium channel blockers that can be selected from the group consisting of, but not limited to, amlodipine, aranidipine, azelnidipine, barnidipine, benidipine, felodipine, lacidipine, lercanidipine, manidipine, nicardipine, nifedipine, nilvadipine, nimodipine, nisoldipine, nitrendipine, pranidipine, preferably amlodipine, and any pharmaceutically acceptable salts or esters thereof and any combinations thereof.

Preferably the term RAAS inhibitor as used in the present invention means angiotensin II receptor antagonist, more preferably valsartan or telmisartan or any pharmaceutically acceptable salts or esters thereof.

The term HMG-CoA reductase inhibitor as used in the present invention can include, but is not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof.

Moreover, the term HMG-CoA reductase inhibitor as used in the present invention can further include one or more combination with other active substance such as for example, but not limited to, combination with cholesterol absorption inhibitor such as ezetimibe, combination with calcium channel blockers, such as for example dihydropiridine calcium channel blockers that can be selected from the group consisting of, but not limited to, amlodipine, aranidipine, azelnidipine, barnidipine, benidipine, felodipine, lacidipine, lercanidipine, manidipine, nicardipine, nifedipine, nilvadipine, nimodipine, nisoldipine, nitrendipine, pranidipine, preferably amlodipine, and any pharmaceutically acceptable salts or esters thereof and any combinations thereof.

Preferably the term HMG-CoA reductase inhibitor as used in the present invention means fluvastatin or atorvastatin or any pharmaceutically acceptable salts or esters thereof.

In one embodiment, the present invention relates to the pharmaceutical composition comprising at least one renin-angiotensin-aldosterone system inhibitor selected from the group consisting of, but not limited to, renin inhibitor, angiotensin-converting enzymeinhibitor and angiotensin II receptor antagonist and any combinations thereof in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

In a preferred embodiment, the present invention relates to the pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a subtherapeutic daily dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

The term anti-inflammatory agent as used in the present invention can include, but is not limited to, classic non-steroidal antiinflammatory agents (NSAIDS), such as for example acetylsalicyclic acid, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nabumetone, acetaminophen and any pharmaceutically acceptable salts thereof; COX-2 inhibitors, such as for example nimesulide, flosulid, celecoxib, rofecoxib, parecoxib sodium, valdecoxib, etoricoxib, etodolac, meloxicam and any pharmaceutically acceptable salts thereof; glucocorticoids, such as for example hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, rapamycin and any pharmaceutically acceptable salts thereof; resveratrol and any analogues of these agents. Preferably anti-inflammatory agent can be selected from the group consisting of, but not limited to, acetylsalicyclic acid, ketoprofen, ibuprofen, naproxen, celecoxib, rofecoxib, meloxicam, hydrocortisone, cortisone, prednisone, prednisolone, betamethasone, dexamethasone, resveratrol and any pharmaceutically acceptable salts thereof and/or any analogues, more preferably acetylsalicyclic acid, ibuprofen, celecoxib, hydrocortisone, dexamethasone, resveratrol and any pharmaceutically acceptable salts thereof and/or any analogues of these agents, and even more preferably acetylsalicyclic acid and resveratrol and any pharmaceutically acceptable salts thereof and/or any analogues thereof.

In a preferred embodiment of the invention, the anti-inflammatory agent is present in the pharmaceutical composition in the efficient amount to reduce inflammation.

The term antioxidant as used in the present invention can include, but is not limited to, butylatedhydroxyanisole, butylatedhydroxytoluene, malic acid, ascorbylpalmitate, sodium ascorbate, sodium metabisulphite, propyl gallate, beta-carotene, ascorbic acid, sodium ascorbyl phosphate, magnesium ascorbyl phosphate, ascorbic acid-2-glycoside, ascorbylpalmitate, ascorbyl stearate, α-lipoic acid, glutathione, coenzyme Q10, tocopherol, tocopherol acetate, retinol, retinol palmitate, genistein, quercetin, epigallocatechin, epigallocatechingallate, gallocatechingallate, sylibin, diosmetin, kaempferol, epicatechin, galangin, indolic acid, γ-linolenic acid, linoleic acid, chlorogenic acid, tocotrienol, astaxanthin, and any pharmaceutically acceptable salts thereof and/or any analogues thereof. Preferably, the antioxidant can be selected from the group consisting of, but not limited to, ascorbic acid, sodium ascorbyl phosphate, coenzyme Q10, magnesium ascorbyl phosphate, ascorbic acid-2-glycoside, butylatedhydroxyanisole, chlorogenic acid, epigallocatechingallate, indolic acid, α-lipoic acid and any pharmaceutically acceptable salts thereof and/or any analogues thereof, more preferably ascorbic acid, sodium ascorbyl phosphate, coenzyme Q10, magnesium ascorbyl phosphate, ascorbic acid-2-glycoside, butylatedhydroxyanisole and any pharmaceutically acceptable salts thereof and/or any analogues thereof and even more preferably coenzyme Q10 and any pharmaceutically acceptable salts thereof and/or any analogue thereof.

In a preferred embodiment of the invention, the antioxidant is present in the pharmaceutical composition in the efficient amount to inhibit oxidation.

According to the present invention, the term subtherapeutic daily dose relates to a dose that does not lower cholesterol level and blood pressure as defined above, therefore the beneficial effects at this dose are attributed solely/purely to the pleotropic effects of renin-angiotensin-aldosterone system inhibitor and HMG-CoA reductase inhibitor. Preferably, the subtherapeutic daily dose is between 1 and 50%, more preferably between 1 and 25% of daily recommended therapeutic dose for particular active substance. A subtherapeutic daily dose does not produce side-effects which are important limitations of therapeutic dosages particularly for long term usage during which known and still unknown complications or side-effects could occur. It is well known that side-effects are related to the dose of the used drug being more frequent at higher dosages.

According to the state of the art, the starting recommended daily dose for valsartan is 80 mg and the starting recommended daily dose for telmisartan is 40 mg, therefore the term subtherapeutic daily dose according to the present invention preferably means between 1 and 40 mg if valsartan or any pharmaceutically acceptable salts or esters thereof is used as RAAS inhibitor and preferably between 1 and 20 mg if telmisartan or any pharmaceutically acceptable salts or esters thereof is used as RAAS inhibitor.

According to the state of the art, the starting recommended daily dose for fluvastatin sodium is 20 mg and the starting recommended daily dose for atorvastatin calcium is 10 mg, therefore the term subtherapeutic daily dose according to the present invention means preferably between 1 and 10 mg if fluvastatin or any pharmaceutically acceptable salts or esters thereof is used as HMG-CoA reductase inhibitor and preferably between 1 and 5 mg if atorvastatin or any pharmaceutically acceptable salts or esters thereof is used as HMG-CoA reductase inhibitor.

In a preferred embodiment, the present invention relates to the pharmaceutical composition comprising at least one renin-angiotensin-aldosterone system inhibitor in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects.

In another preferred embodiment, the present invention relates to the pharmaceutical composition comprising at least one renin-angiotensin-aldosterone system inhibitor selected from the group consisting of, but not limited to, renin inhibitor, angiotensin-converting enzyme inhibitor and angiotensin II receptor antagonist and any combinations thereof in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects.

In another preferred embodiment, the present invention relates to the pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a daily dose between 1 and 50%, preferably between 1 and 25% of daily recommended therapeutic dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and optionally at least one other active agent selected from the group consisting of an anti-inflammatory agent, an antioxidant or any mixtures thereof for use in prevention, reduction or reversal of arterial ageing in apparently healthy subjects.

The term apparently healthy subject according to the present invention relates to a subject having a very low cardiovascular risk as defined above and who does preferably not have (manifested) cardiovascular disorders and who preferably does not have disorders which importantly influence the functional capacity of different tissues/organs or the whole body. The term cardiovascular disorder (CVD) according to the present invention refers to the cardiovascular disorder or event such as for example ischemic heart disease, carotid and intracerebral artery disease, peripheral arterial disease, aortic aneurism and similar, and any combinations thereof. Preferably CVD refers to myocardial infarction, stroke, dementia, critical limb ischemia, aortic aneurism and any combinations thereof, more preferably to myocardial infarction, stroke, vascular dementia and any combinations thereof. Furthermore, it was unexpectedly found out that the effect of prevention, reduction or reversal of arterial ageing in apparently healthy subjects when using the pharmaceutical composition according to the present invention is surprisingly achieved after treatment defined as a treatment-period, that can last for at least 1 week, between about 2 weeks to about 3 months, preferably between about 2 weeks to about 2 months and more preferably between about 2 weeks to about 1 month.

Therefore, in another preferred embodiment, the present invention relates to a pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects in a treatment-period lasting at least 1 week, between about 2 weeks to about 3 months, preferably between about 2 weeks to about 2 months and more preferably between about 2 weeks to about 1 month.

In another preferred embodiment, the present invention relates to a pharmaceutical composition comprising at least one renin-angiotensin-aldosterone system inhibitor in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects in a treatment-period lasting between at least one week, about 2 weeks to about 3 months, preferably between about 2 weeks to about 2 months and more preferably between about 2 weeks to about 1 month.

In another preferred embodiment, the present invention relates to a pharmaceutical composition comprising at least one renin-angiotensin-aldosterone system inhibitor selected from the group consisting of, but not limited to, renin inhibitor, angiotensin-converting enzyme inhibitor and angiotensin II receptor antagonist and any combinations thereof in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects in a treatment-period lasting at least 1 week, between about 2 weeks to about 3 months, preferably between about 2 weeks to about 2 months and more preferably between about 2 weeks to about 1 month.

In another preferred embodiment, the present invention relates to a pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a subtherapeutic daily dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects in a treatment-period lasting at least 1 week, between about 2 weeks to about 3 months, preferably between about 2 weeks to about 2 months and more preferably between about 2 weeks to about 1 month.

In another preferred embodiment, the present invention relates to a pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and optionally at least one other active agent selected from the group consisting of an anti-inflammatory agent, an antioxidant or any mixtures thereof for use in prevention, reduction or reversal of arterial ageing in apparently healthy subjects in a treatment-period lasting at least 1 week, between about 2 weeks to about 3 months, preferably between about 2 weeks to about 2 months and more preferably between about 2 weeks to about 1 month.

The above mentioned surprising effects of the present invention were determined in a double-blind study wherein 40 apparently healthy subjects were randomly assigned to treatment (fluvastatin sodium 10 mg/valsartan 20 mg daily, 1 month—30 days) or placebo. The main functional and morphological characteristics of arteries were tested by measurement of flow-mediated dilatation of brachial artery (FMD), pulse-wave velocity (PWV) and β-stiffness of carotid artery once at baseline and after 30 days. All parameters of arterial function were significantly improved after 30 days of treatment:

a) FMD increased by 275.7% (p<0.001),
b) PWV decreased by 10.1% (p<0.01) and
c) β-stiffness decreased by 16.8% (p<0.001).

Said beneficial arterial characteristics were not accompanied by any substantial changes in lipids or blood pressure. Moreover, unexpectedly the substantial synergistic improvement was achieved in comparison to the group administering only angiotensin II receptor antagonist or only HMG CoA reductase inhibitor.

Furthermore, the inventors observed substantial long term persistence of beneficial arterial characteristics. Thus, it was unexpectedly found out that the synergistic effect on arterial ageing when administering the pharmaceutical composition according to the present invention surprisingly persisted in a substantial amount even approximately 1 month, 3, 4, 5, 6, 7, 8 or 12 months, preferably between 6 and 12 months, after discontinuation of treatment. The period without any treatment according to the present invention and wherein the beneficial arterial characteristics are still present is named as the rest-period. One of the aims of the rest-period is to prevent the occurrence of ‘resistance’ to therapy leading to decreased efficacy after certain time. If any inhibitory process is induced by treatment which seems to be logical it would be diminished during the rest period. That means that repeating of treatment would not result in a decreased efficacy but rather in a similar or even higher efficacy. Based on that assumption one could predict that a very long term of use (decades) could be possible without significantly lost efficacy of treatment. Another important aims of rest-period are higher compliance of patients and fewer side effects.

Therefore, in another preferred embodiment, the present invention relates to the pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects having at least one treatment period and at least on erest period characterized in that the rest-period is approximately 1 month, 3, 4, 5, 6, 7, 8 or 12 months, preferably between 6 and 12 months.

In another preferred embodiment, the present invention relates to a pharmaceutical composition comprising at least one renin-angiotensin-aldosterone system inhibitor in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects characterized in that the rest-period is approximately 1 month, 3, 4, 5, 6, 7, 8 or 12 months, preferably between 6 and 12 months.

In another preferred embodiment, the present invention relates to a pharmaceutical composition comprising at least one renin-angiotensin-aldosterone system inhibitor selected from the group consisting of, but not limited to, renin inhibitor, angiotensin-converting enzymeinhibitor and angiotensin II receptor antagonist and any combinations thereof and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects characterized in that the rest-period is approximately 1 month, 3, 4, 5, 6, 7, 8 or 12 months, preferably between 6 and 12 months.

In another preferred embodiment, the present invention relates to a pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a subtherapeutic daily dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects characterized in that the rest-period is approximately 1 month, 3, 4, 5, 6, 7, 8 or 12 months, preferably between 6 and 12 months.

In another preferred embodiment, the present invention relates to a pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and optionally at least one other active agent selected from the group consisting of an anti-inflammatory agent, an antioxidant or any mixtures thereof for use in prevention, reduction or reversal of arterial ageing in apparently healthy characterized in that the rest-period is approximately 1 month, 3, 4, 5, 6, 7, 8 or 12 months, preferably between 6 and 12 months.

In extension of the above mentioned study, the presence of the beneficial arterial characteristics was measured after discontinuation of treatment. Surprisingly it was found out that the beneficial arterial characteristics measured in the same observed group of subjects were still present in substantial amounts even after up to 12 months. For example, after 7 months of discontinuation the beneficial arterial characteristics were still present at the following percentage of initial improvement achieved after 1 month (30 days) of treatment disclosed above:

a) FMD still increased by 81.0% (p<0.05),
b) PWV still decreased by 70.0% (p<0.05) and
c) β-stiffness still decreased by 68.0% (p<0.01).

Moreover, again unexpectedly the substantial synergistic improvement was achieved in comparison to the group administering only angiotensin II receptor antagonist or only HMG CoA reductase inhibitor.

In another preferred embodiment, the present invention relates to a specific, original approach for implementation of the above mentioned obtained beneficial arterial characteristics by the following treatment regime: one treatment-period followed by one rest-period represents, i.e. one intervention-cycle that can be repeated at least 3, 4 or 5 times.

Therefore, in another preferred embodiment, the present invention relates to a pharmaceutical composition comprising at least one renin-angiotensin-aldosterone system inhibitor in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times.

In another preferred embodiment, the present invention relates to a pharmaceutical composition comprising at least one renin-angiotensin-aldosterone system inhibitor in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times.

In another preferred embodiment, the present invention relates to a pharmaceutical composition comprising at least one renin-angiotensin-aldosterone system inhibitor selected from the group consisting of, but not limited to, renin inhibitor, angiotensin-converting enzyme inhibitor and angiotensin II receptor antagonist and any combinations thereof in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times.

In another preferred embodiment, the present invention relates to a pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a subtherapeutic daily dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times.

In another preferred embodiment, the present invention relates to a pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3, 4 or 5 times.

It is well known that neither renin-angiotensin-aldosterone system inhibitor nor HMG CoA reductase inhibitors at low doses affect the plasma concentration of high sensitivity C-Reactive Protein (hsCRP) which is a well known indicator for a risk factor for cardiovascular disorders. The inventors of the present application surprisingly found out that the subtherapeutical daily dose combination of at least one angiotensin II receptor antagonist and at least one HMG CoA reductase inhibitor (1 month/30 days treatment) substantially decrease, by approximately 20%, the plasma concentration of hsCRP.

The authors of the present invention surprisingly found out that the pharmaceutical composition according to the present invention, which substantially decrease arterial age, additionally reveal two unexpected findings:
1) Efficacy of treatment is similar even in older subjects than in much younger subjects.
2) Older subjects still have a substantial capacity for improvement of arterial functions or in other words for reduction of arterial age.

Therefore, it could be concluded that the use of pharmaceutical composition according to the present invention is efficient in a wide range of years/ages, and that it could be started later in life (for example at age 50 or even later) with the same or substantially the same expected rate of success.

It is well known from the state of the art that functional and morphological properties of arterial wall have high predictive values and have important causal role on worsening or occurrence of cardiovascular disorders. At the same time it is well known that age (chronological or biological age) is one of the most important risk factor for the worsening or occurrence of cardiovascular disorders. On the other hand, it is also well known that cardiovascular disorders themselves simultaneously accelerate the arterial ageing and biological ageing. Hence, it is clear from the above that arterial age is a risk factor for cardiovascular disorders. Therefore, the reduction and reversal of arterial age results in decreased risk for cardiovascular disorders. Putting all these facts together, it can be concluded that improvement in arterial wall properties should be pivotal aim in decreasing both arterial age and risk for cardiovascular disorder. By said approach simultaneous achievement of two tremendously important aims:
decreasing (biological) arterial age and
decreasing occurrence of cardiovascular disorders
   are assured and thereby extending the duration and quality of life.

Therefore, in one embodiment, the present invention relates to a pharmaceutical composition comprising at least one renin-angiotensin-aldosterone system inhibitor in a subtherapeutic daily dose and at least one HMG-CoA reductase inhibitor in a subtherapeutic daily dose for use in decreasing occurrence of cardiovascular disorders in apparently healthy subjects.

In a preferred embodiment, the present invention relates to a pharmaceutical composition comprising at least one renin-angiotensin-aldosterone system inhibitor in a daily dose between 1 and 50%, preferably between 1 and 25% of daily recommended therapeutic dose and at least one HMG-CoA reductase inhibitor in a daily dose between 1 and 50%, preferably between 1 and 25% of daily recommended therapeutic dose and optionally at least one other active agent selected from the group consisting of an anti-inflammatory agent, an antioxidant or any mixtures thereof for use in decreasing occurrence of cardiovascular disorders in apparently healthy subjects.

In another preferred embodiment, the present invention relates to a pharmaceutical composition comprising at least one renin-angiotensin-aldosterone system inhibitor selected from the group consisting of, but not limited to, renin inhibitor, angiotensin-converting enzymeinhibitor and angiotensin II receptor antagonist and any combinations thereof in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in decreasing occurrence of cardiovascular disorders in apparently healthy subjects.

In another preferred embodiment, the present invention relates to a pharmaceutical composition comprising at least one angiotensin II receptor antagonist, selected from the group consisting of, but not limited to, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof, in a subtherapeutic daily dose and at least one HMG-CoA reductase inhibitor, selected from the group consisting of, but not limited to, mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof, in a subtherapeutic daily dose and optionally at least one other active agent selected from the group consisting of an anti-inflammatory agent, an antioxidant or any mixtures thereof for use in decreasing occurrence of cardiovascular disorders in apparently healthy subjects.

In another preferred embodiment, the present invention relates to a pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and optionally at least one other active agent selected from the group consisting of an anti-inflammatory agent, an antioxidant or any mixtures thereof for use in decreasing occurrence of cardiovascular disorders in apparently healthy subjects.

In one embodiment, the present invention relates to the pharmaceutical composition comprising at least one renin-angiotensin-aldosterone system inhibitor as defined above in a subtherapeutic daily dose and at least one HMG-CoA reductase inhibitor as defined above in a subtherapeutic daily dose, and optionally at least one other active agent selected from the group consisting of an anti-inflammatory agent as defined above, an antioxidant as defined above or any mixtures thereof.

In a preferred embodiment, the present invention relates to the pharmaceutical composition comprising at least one angiotensin-II-receptor blocker as defined above in a subtherapeutic daily dose and at least one HMG-CoA reductase inhibitor as defined above in a subtherapeutic daily dose, and optionally at least one other active agent selected from the group consisting of an anti-inflammatory agent as defined above, an antioxidant as defined above or any mixtures thereof.

Accordingly, in another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and acetylsalicylic acid and coenzyme Q10 in any pharmaceutically acceptable form.

In another preferred embodiment, the pharmaceutical composition comprises valsartan or any pharmaceutically acceptable salts thereof in a daily dose between 1 and 40 mg and fluvastatin or any pharmaceutically acceptable salts thereof in a daily dose between 1 and 10 mg and acetylsalicylic acid and coenzyme Q10 in any pharmaceutically acceptable form.

In another preferred embodiment, the pharmaceutical composition comprises valsartan or any pharmaceutically acceptable salts thereof in a subtherapeutic daily dose and fluvastatin or any pharmaceutically acceptable salts thereof in a subtherapeutic daily dose and resveratrol or any pharmaceutically acceptable salts thereof and coenzyme Q10 in any pharmaceutically acceptable form.

In another preferred embodiment, the pharmaceutical composition comprises valsartan or any pharmaceutically acceptable salts thereof in a daily dose between 1 and 40 mg and fluvastatin or any pharmaceutically acceptable salts thereof in a daily dose between 1 and 10 mg and resveratrol or any pharmaceutically acceptable salts thereof and coenzyme Q10 in any pharmaceutically acceptable form.

In another preferred embodiment, the pharmaceutical composition comprises valsartan or any pharmaceutically acceptable salts thereof in a subtherapeutic daily dose and fluvastatin or any pharmaceutically acceptable salts thereof in a subtherapeutic daily dose and acetylsalicylic acid.

In another preferred embodiment, the pharmaceutical composition comprises valsartan or any pharmaceutically acceptable salts thereof in a daily dose between 1 and 40 mg and fluvastatin or any pharmaceutically acceptable salts thereof in a daily dose between 1 and 10 mg and acetylsalicylic acid.

In another preferred embodiment, the pharmaceutical composition comprises valsartan or any pharmaceutically acceptable salts thereof in a subtherapeutic daily dose and fluvastatin or any pharmaceutically acceptable salts thereof in a subtherapeutic daily dose and resveratrol or any pharmaceutically acceptable salts thereof.

In another preferred embodiment, the pharmaceutical composition comprises valsartan or any pharmaceutically acceptable salts thereof in a daily dose between 1 and 40 mg and fluvastatin or any pharmaceutically acceptable salts thereof in a daily dose between 1 and 10 mg and resveratrol or any pharmaceutically acceptable salts thereof.

In another preferred embodiment, the present invention is the pharmaceutical composition comprises valsartan or any pharmaceutically acceptable salts thereof in a subtherapeutic daily dose and fluvastatin or any pharmaceutically acceptable salts thereof in a subtherapeutic daily dose and coenzyme Q10 in any pharmaceutically acceptable form.

In another preferred embodiment, the pharmaceutical composition comprises valsartan or any pharmaceutically acceptable salts thereof in a daily dose between 1 and 40 mg and fluvastatin or any pharmaceutically acceptable salts thereof in a daily dose between 1 and 10 mg and coenzyme Q10 in any pharmaceutically acceptable form.

Accordingly, in another preferred embodiment, the pharmaceutical composition comprises valsartan or any pharmaceutically acceptable salts thereof in a subtherapeutic daily dose and fluvastatin or any pharmaceutically acceptable salts thereof in a subtherapeutic daily dose.

In another preferred embodiment, the pharmaceutical composition comprises valsartan or any pharmaceutically acceptable salts in a daily dose between 1 and 40 mg and fluvastatin or any pharmaceutically acceptable salts thereof in a daily dose between 1 and 10 mg.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and acetylsalicylic acid and coenzyme Q10 in any pharmaceutically acceptable for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and acetylsalicylic acid and coenzyme Q10 in any pharmaceutically acceptable for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and resveratrol or any pharmaceutically acceptable salts thereof and coenzyme Q10 in any pharmaceutically acceptable form for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and resveratrol or any pharmaceutically acceptable salts thereof and coenzyme Q10 in any pharmaceutically acceptable form for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and acetylsalicylic acid for use in prevention, reduction or reversal of arterial ageing in apparently healthy subjects.

In another preferred embodiment, the present invention relates to the pharmaceutical composition comprising valsartan or any pharmaceutically acceptable salts in a daily dose between 1 and 40 mg and fluvastatin or any pharmaceutically acceptable salts thereof in a daily dose between 1 and 10 mg and acetylsalicylic acid for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and resveratrol or any pharmaceutically acceptable salts thereof for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and resveratrol or any pharmaceutically acceptable salts thereof for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose and coenzyme Q10 in any pharmaceutically acceptable form for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg and coenzyme Q10 in any pharmaceutically acceptable form for use in prevention, reduction or reversal of arterial ageing in apparently healthy subjects.

In another preferred embodiment, the present invention relates to the pharmaceutical composition comprising valsartan or any pharmaceutically acceptable salts thereof in a subtherapeutic daily dose and fluvastatin or any pharmaceutically acceptable salts thereof in a subtherapeutic daily dose for use in prevention, reduction or reversal of arterial ageing in apparently healthy subjects.

In another preferred embodiment, the present invention relates to the pharmaceutical composition comprising valsartan or any pharmaceutically acceptable salts thereof in a daily dose between 1 and 40 mg and fluvastatin or any pharmaceutically acceptable salts thereof in a daily dose between 1 and 10 mg for use in prevention, reduction or reversal of arterial ageing in apparently healthy subjects.

The term pharmaceutical composition according to the present invention may mean that each component of the composition is administered to the subject separately in an individual dosage form simultaneously, separately or sequentially in any order. The present invention furthermore relates to a commercial package comprising the pharmaceutical composition according to the present invention together with instructions for simultaneous, separate or sequential use.

Alternatively the term pharmaceutical composition according to the present invention may mean that all or just some components of the composition are administered to the subject in the same unit dosage form. The combination of two or more active agents in the same pharmaceutical composition provides the additional advantage of reducing the frequency of administration of a dosage, thereby increasing the safety of the therapy and it is more patient friendly.

Therefore, in a preferred embodiment, the present invention relates to the pharmaceutical composition according to the present invention together with one or more pharmaceutically acceptable excipient. The term ‘pharmaceutically acceptable’ as employed herein refers to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio. The term ‘pharmaceutically acceptable excipient’ means a component of a pharmaceutical product that is not an active ingredient. Useful pharmaceutically acceptable excipients of the present invention include, but are not limited to, diluents, disintegrants, binders, lubricants, surfactants, pH modifiers, antiadherants, pigments, colorants and the like, and any combinations thereof.

The pharmaceutical composition according to the present invention may be administered to the subject by any known route of administration such as for example peroral (mouth), topical (skin), parenteral (skin or mucous membrane), transmucosal (nasal, buccal/sublingual, vaginal, ocular, rectal) or inhalation. The pharmaceutical composition according to the present invention may be useful for immediate-, delayed-, modified-, sustained-, extended-, pulsed-, continuous- or controlled-release applications. The pharmaceutical composition according to the present invention may be prepared by any process known from the state of the art.

The pharmaceutical composition according to the present invention suitable for peroral administration may take the form of, but is not limited to, solution, suspension, emulsion, tablet, pill, gel, syrup, elixir, capsule, powder, liquid or solid crystal, paste, and the like.

The pharmaceutical composition according to the present invention suitable for topical administration may take the form of, but are not limited to, cream, gel, liniment or balm, lotion, ointment, ear drops, eye drops, skin patch and the like.

The pharmaceutical composition according to the present invention suitable for parenteral administration may refer to modes of administration which include, but are not limited to, intradermal, intraosseous, intravenous, intramuscular, intraperitoneal, intrasternal, subcutaneous and intraarticular injection and infusion.

The pharmaceutical composition according to the present invention suitable for inhalation may take the form of, but is not limited to, aerosol, inhaler, nebulizer, vaporizer and the like.

The pharmaceutical composition according to the present invention may be in the form of suppositories such as for example rectal or vaginal suppositories.

To conclude, the inventors of the present application surprisingly found out that arterial ageing (in particular typical functional and morphological characteristics of arterial wall that can be measured by standard and widely used methods) can be prevented, reduced or reversed by administering the pharmaceutical composition according to the present invention. The achieved beneficial arterial characteristics were not accompanied by the primary action of renin-angiotensin-aldosterone system inhibitor or HMG CoA reductase inhibitor i.e. reduction of plasma lipids and blood pressure. Unexpectedly, the improvement in age-related characteristics in the observed population was achieved already after short-term treatment (for example at least one month) and again, unexpectedly, persists at substantial level approximately 12 months after discontinuation of treatment. The vascular effects were followed by a substantial decrease of hsCRP, which is a well-known marker of (intraarterial) inflammation and well-known marker of cardiovascular risk. The pharmaceutical composition according to the present invention reveals several synergistic effects in comparison with both classes of drugs alone. The unique efficacy profile of said composition of the present invention allows a cyclic treatment consisting of a short term treatment-period followed by a long term rest-period during which beneficial arterial characteristics are still present.

In particular, the present invention comprises the following preferred embodiments:

1. A pharmaceutical combination composition comprising at least one angiotensin II receptor antagonist in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

2. pharmaceutical combination composition comprising at least one angiotensin II receptor antagonist in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects wherein one intervention-cycle is repeated at least 3 to 5 times.

3. The pharmaceutical combination composition according to items 1 and 2 wherein the subtherapeutic daily dose is less than 50%, preferably less than 25% of daily recommended therapeutic dose.

4. The pharmaceutical combination composition according to anyone of items 1 to 3 wherein angiotensin II receptor antagonist is selected from the group consisting of losartan, irbesartan, olmesartan, candesartan, valsartan and telmisartan and any pharmaceutically acceptable salts thereof

5. The pharmaceutical combination composition according to item 4 wherein angiotensin II receptor antagonist is valsartan.

6. The pharmaceutical combination composition according to anyone of items 1 to 3 wherein HMG-CoA reductase inhibitor is selected from the group consisting of mevastatin, pitavastatin, lovastatin, simvastatin, pravastatin, fluvastatin, atorvastatin, cerivastatin, and rosuvastatin and any pharmaceutically acceptable salts thereof

7. The pharmaceutical combination composition according to item 6 wherein HMG-CoA reductase inhibitor is fluvastatin sodium.

8. The pharmaceutical combination composition according to anyone of items 1 to 3 wherein anti-inflammatory agent is selected from the group consisting of acetylsalicylic acid and resveratrol.

9. The pharmaceutical combination composition according to anyone of items 1 to 3 wherein antioxidant is coenzyme Q10 or any analogues thereof

10. The pharmaceutical combination composition according to anyone of items 1 to 9 wherein one intervention-cycle consists of one treatment-period lasting between about 2 weeks to about 3 months, preferably between about 2 weeks to about 2 months and more preferably between about 2 weeks to about 1 month and one rest-period lasting 12 months, preferably between 6 and 12 months.

11. The pharmaceutical combination composition comprising valsartan or any pharmaceutically acceptable salts thereof in a subtherapeutic daily dose and fluvastatin or any pharmaceutically acceptable salts thereof in a subtherapeutic daily dose and optionally acetylsalicylic acid, resveratrol and/or coenzyme Q10 in any pharmaceutically acceptable form.

12. The pharmaceutical combination composition comprising valsartan or any pharmaceutically acceptable salts thereof in a subtherapeutic daily dose and fluvastatin or any pharmaceutically acceptable salts thereof in a subtherapeutic daily dose and optionally acetylsalicylic acid, resveratrol and/or coenzyme Q10 in any pharmaceutically acceptable form for use in prevention, reduction or reversal of arterial ageing in apparently healthy subjects.

13. The pharmaceutical combination composition according to items 11 and 12 wherein the subtherapeutic daily dose is less than 50%, preferably less than 25% of daily recommended therapeutic dose.

14. The pharmaceutical combination composition according to any one of items 1 to 13 further comprising one or more pharmaceutically acceptable excipient.

Further, the present invention comprises the following preferred embodiments:

1. A pharmaceutical combination composition comprising at least one renin-angiotensin-aldosterone system inhibitor, selected from the group consisting of, renin inhibitor, angiotensin-converting enzymeinhibitor and angiotensin II receptor antagonist and any combinations thereof and at least one HMG-CoA reductase inhibitor in a subtherapeutic daily dose and optionally at least one other active agent selected from the group consisting of an anti-inflammatory agent, an antioxidant or any mixtures thereof for use in prevention, reduction or reversal of arterial ageing in apparently healthy subjects.

2. A pharmaceutical combination composition comprising at least one renin-angiotensin-aldosterone system inhibitor, selected from the group consisting of, renin inhibitor, angiotensin-converting enzymeinhibitor and angiotensin II receptor antagonist and any combinations thereof in a subtherapeutic daily dose and at least one HMG-CoA reductase inhibitor in a subtherapeutic daily dose and optionally at least one other active agent selected from the group consisting of an anti-inflammatory agent, an antioxidant or any mixtures thereof for use in decreasing occurrence of cardiovascular disorders in apparently healthy subjects.

3. A pharmaceutical combination composition according to items 1 and 2 wherein one intervention-cycle is repeated at least 3 to 5 times.

4. The pharmaceutical combination composition according to items 1 to 3 wherein the subtherapeutic daily dose is between 1 and 50%, preferably between 1 and 25% of daily recommended therapeutic dose.

5. The pharmaceutical combination composition according to anyone of items 1 to 4 wherein renin-angiotensin-aldosterone system inhibitor is angiotensin II receptor antagonist, selected from the group consisting of, azilsartan, losartan, eprosartan, irbesartan, olmesartan, candesartan, valsartan and telmisartan and any pharmaceutically acceptable salts or esters thereof, preferably azilsartan, losartan, telmisartan, olmesartan, candesartan and valsartan and any pharmaceutically acceptable salts or esters thereof, more preferably losartan, telmisartan, azilsartan, candesartan and valsartan and any pharmaceutically acceptable salts or esters thereof and even more preferably valsartan and telmisartan and any pharmaceutically acceptable salts or esters thereof.

6. The pharmaceutical combination composition according to item 5 wherein angiotensin II receptor antagonist is valsartan or any pharmaceutically acceptable salts.

7. The pharmaceutical combination composition according to anyone of items 1 to 4 wherein HMG-CoA reductase inhibitor is selected from the group consisting of mevastatin, pitavastatin, lovastatin, simvastatin, pravastatin, fluvastatin, atorvastatin, cerivastatin, and rosuvastatin and any pharmaceutically acceptable salts or esters thereof, preferably simvastatin, fluvastatin, atorvastatin and rosuvastatin and any pharmaceutically acceptable salts or esters thereof, more preferably fluvastatin, atorvastatin and rosuvastatin and any pharmaceutically acceptable salts or esters thereof and even more preferably fluvastatin and atorvastatin and any pharmaceutically acceptable salts or esters thereof

8. The pharmaceutical combination composition according to item 7 wherein HMG-CoA reductase inhibitor is fluvastatin sodium.

9. The pharmaceutical combination composition according to anyone of items 1 to 8 wherein anti-inflammatory agent is selected from the group consisting of acetylsalicylic acid and resveratrol.

10. The pharmaceutical combination composition according to anyone of items 1 to 9 wherein antioxidant is coenzyme Q10 or any analogues thereof

11. The pharmaceutical combination composition according to anyone of items 1 to 10 wherein one intervention-cycle consists of one treatment-period lasting between about 2 weeks to about 3 months, preferably between about 2 weeks to about 2 months and more preferably between about 2 weeks to about 1 month and one rest-period lasting 12 months, preferably between 6 and 12 months.

12. The pharmaceutical combination composition comprising valsartan or any pharmaceutically acceptable salts thereof in a daily dose between 1 and 40 mg and fluvastatin or any pharmaceutically acceptable salts thereof in a daily dose between 1 and 10 mg and optionally acetylsalicylic acid, resveratrol and/or coenzyme Q10 in any pharmaceutically acceptable form.

13. The pharmaceutical combination composition comprising valsartan or any pharmaceutically acceptable salts thereof in a daily dose between 1 and 40 mg and fluvastatin or any pharmaceutically acceptable salts thereof in a daily dose between 1 and 10 mg and optionally acetylsalicylic acid, resveratrol and/or coenzyme Q10 in any pharmaceutically acceptable form for use in prevention, reduction or reversal of arterial ageing in apparently healthy subjects.

14. The pharmaceutical combination composition comprising valsartan or any pharmaceutically acceptable salts thereof in a daily dose between 1 and 40 mg and fluvastatin or any pharmaceutically acceptable salts thereof in a daily dose between 1 and 10 mg and optionally acetylsalicylic acid, resveratrol and/or coenzyme Q10 in any pharmaceutically acceptable form for use in decreasing occurrence of cardiovascular disorders in apparently healthy subjects.

15. The pharmaceutical combination composition according to any one of items 1 to 13 further comprising one or more pharmaceutically acceptable excipient.

The present invention is illustrated in further detail with reference to the following examples. However, the scope of the present invention is not limited to these examples.

### Example 1

### a) Subjects and Experimental Design

Forty apparently healthy male individuals (42.9±4.2 years) were recruited in double blind, randomized study. Inclusion criteria were:

a) chronological age between 20 and 65 years and
b) no history of cardiovascular disease.

The participants in the study had a Framingham risk factor for a CHD (10 years) of 6.4.

The pharmaceutical combination composition comprising valsartan (as a representative of angiotensin II receptor antagonist) and fluvastatin sodium (as a representative of HMG CoA reductase inhibitor) and following pharmaceutically acceptable excipients microcrystalline cellulose, crospovidone, colloidal anhydrous silica, potassium hydrogen carbonate, magnesium stearate, hydroxypropyl methylcellulose, polyethylene glycol, talc, titanium dioxide and iron oxide was used.

The control group (n=20) received placebo, while the test group (n=20) received subtherapeutic daily dose of valsartan—20 mg daily and subtherapeutic daily dose of fluvastatin sodium—10 mg daily during a period of 1 month—30 days.

All subjects underwent clinical examination, blood pressure measurements (Welch AllynSpiedel&Keller automated sphygmomanometer) and ultrasound measurement of flow-mediated dilatation of brachial artery (FMD), pulse-wave velocity (PWV) and β-stiffness of carotid artery at inclusion (0^{th} day) and after 1 month (30^{th} day) of the study. Fasting blood samples were taken at the beginning and at the end of the study for laboratory analysis. Blood glucose, electrolytes, hsCRP and cholesterol were obtained using the VITRO 5.1 FS Chemical system (Ortho Clinical Diagnostics, Inc.).

### b) Ultrasound Measurements

Ultrasound measurements were performed by a single examiner using AlokaProSound Alpha 10 echo-machine. Endothelial function was measured by means of FMD on the brachial artery according to FMD guidelines (Corretti M C et al. Guidelines for the ultrasound assessment of endothelial-dependent flow-mediated vasodilation of the brachial artery: a report of the International Brachial Artery Reactivity Task Force. J Am Coll Cardiol 2002; 39: 257-65). The echo-machine continuously tracked and recorded the brachial artery diameter. Following the measurement of baseline brachial artery diameter (1 min), the forearm blood pressure cuff was inflated to 50 mmHg above the systolic pressure for 4 min. After the occlusion period, the cuff was rapidly deflated, inducing reactive hyperemia, and the brachial artery diameter was recorded for 3 min. At the end of the measurement, the machine automatically provided the values of FMD.

The measurements of PWV and β-stiffness were performed on the right common carotid artery. The Aloka ultrasound device was also equipped with special software for automatic determination of the PWV and beta-stiffness through the analysis of pulse waves (Carerj S et al. Normal vascular aging evaluated by a new tool: e-tracking. Eur J Echocardiography 2006; suppl: S 49).

### c) Statistical Analysis

All values were expressed as arithmetic mean±SEM and were normally distributed. Differences between values recorded at the beginning (0^{th} day) and at the end of the study (30^{th} day) were determined by one-way analysis of variance (ANOVA). When a significant interaction was present, the Bonferroni post-test was performed. A P-value of less than 0.05 was considered significant. All statistical analyses were performed using Graph Pad Prism 5.0 software.

### d) Results

Characteristics of the individuals at the beginning and at the end of the treatment in both groups are shown in Table 1.

TABLE 1
Subject characteristics in the placebo and in the test group

| | | | Fluvastatin sodium 10 mg/ | | |
|---|---|---|---|---|---|
| | Placebo (n = 20) | | Valsartan 20 mg (n = 20) | | |
| | 0^{th} day | 30^{th} day | 0^{th} day | 30^{th} day | P value |
| Systolic BP (mmHg) | 123.3 ± 1.8 | 120.8 ± 1.5 | 124.4 ± 1.3 | 120.3 ± 1.8 | 0.21 |
| Diastolic BP (mmHg) | 73.5 ± 2.4 | 72.4 ± 2.3 | 75.1 ± 1.4 | 72.2 ± 1.5 | 0.43 |
| Heart rate (b.p.m.) | 61.1 ± 2.2 | 60.3 ± 1.8 | 59.5 ± 2.5 | 59.7 ± 2.0 | 0.95 |
| Total cholesterol | 5.7 ± 0.3 | 5.6 ± 0.3 | 5.6 ± 0.3 | 5.3 ± 0.2 | 0.35 |
| (mmol/l) | | | | | |
| LDL cholesterol | 3.6 ± 0.2 | 3.6 ± 0.3 | 3.7 ± 0.2 | 3.1 ± 0.3 | 0.14 |
| (mmol/l) | | | | | |
| HDL cholesterol | 1.4 ± 0.1 | 1.4 ± 0.1 | 1.4 ± 0.1 | 1.4 ± 0.1 | 0.99 |
| (mmol/l) | | | | | |
| Triglycerides | 1.3 ± 0.1 | 1.4 ± 0.2 | 1.4 ± 0.2 | 1.5 ± 0.2 | 0.87 |
| (mmol/l) | | | | | |
| Plasma glucose | 4.9 ± 0.2 | 4.9 ± 0.2 | 5.1 ± 0.1 | 4.9 ± 0.2 | 0.73 |
| (mmol/l) | | | | | |

All values are expressed as arithmetic mean ± SEM.
BP: blood pressure;
b.p.m.: beats per minute;
LDL: low-density lipoprotein;
HDL: high-density lipoprotein.

No statistically significant differences in the listed parameters (systolic and diastolic blood pressure, heart rate, total cholesterol, LDL cholesterol, HDL cholesterol, triglycerides and glucose concentration) were observed between the placebo and test group.

TABLE 2
Functional and morphological parameters before and after treatment

| | Before | | |
|---|---|---|---|
| | treatment | After treatment | Improvement |
| FMD (%) | 1.71 | 6.43 | +275.7% |
| PWV (m/s) | 5.78 | 5.25 | −10.1% |
| β-stiffness (U) | 7.11 | 6.09 | −16.8% |
| Arterial age according to age- | 45.0 | 34.0 | −11.0 |
| related normogram (years) | | | |

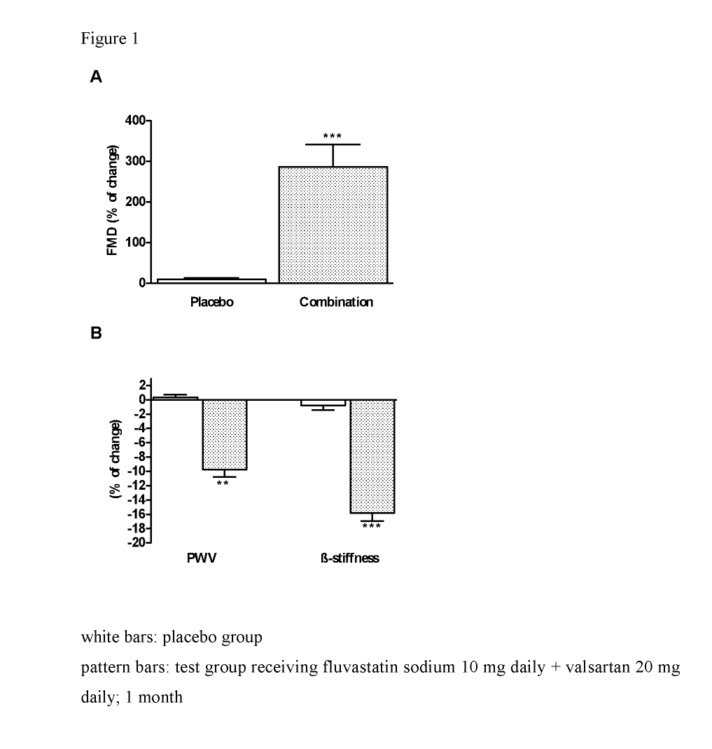

The results presented in Table 2 and FIG. 1 show that FMD increased by 275.7% (P<0.001; FIG. 1A), PWV decreased by 10.1% (P<0.01; FIG. 1B) and β-stiffness of the carotid artery decreased by 16.8% (P<0.001; FIG. 1B) after 1 month treatment period. The substantial improvement was observed in each subject of the test group in all measured ultrasound parameters. No significant changes in described parameters in the placebo group throughout the study were observed.

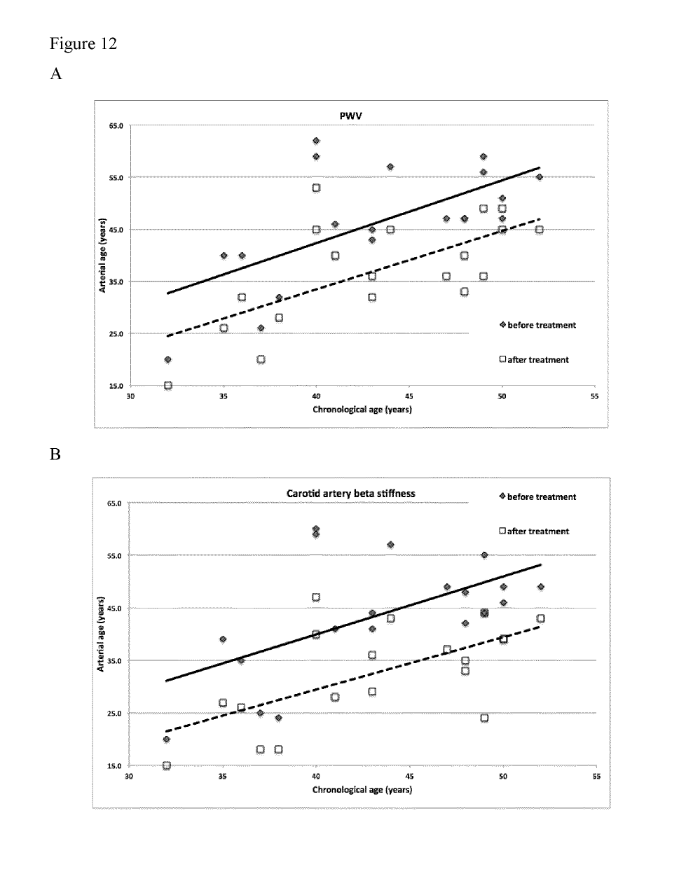

The results presented in FIGS. 12A and 12B show substantial improvement in PWV and β-stiffness after 1 month treatment corresponding to substantial decrease of calculated arterial age by using age-related normograms (according to the methods described in Jurasic M J et al. Beta stiffness—setting age standards. Acta Clin Croat 2009; 48:253-8; and Carerj S. Normal vascular ageing evaluated by a new tool: e-tracking. Eur J Echocardiography 2006; suppl 1: S49.). Overall, 1 month treatment with combination of valsartan—20 mg daily and fluvastatin sodium—10 mg daily results in substantial decrease of arterial age. Moreover, the results reveal two unexpected findings:
1) Efficacy of treatment is similar even in older subjects as in much younger subjects.
2) Older subjects still have a substantial capacity for improvement of arterial functions or in other words for reduction of arterial age.

Therefore, it could be concluded that the use of pharmaceutical combination composition according to the present invention is efficient in wide range of years, and that it could be started later in life (for example at age 50 or even later) with the same expected rate of success.

### Example 2

In the same participants as in Example 1 the levels of hsCRP by standard method using Vitro 5.1 FS Chemical System (Ortho Clinical Diagnostics, Inc).

TABLE 3
hs-CRP values before and after treatment

| | Before | After | |
|---|---|---|---|
| | treatment | treatment | Improvement |
| hsCRP | 1.34 | 1.10 | −21.8% |

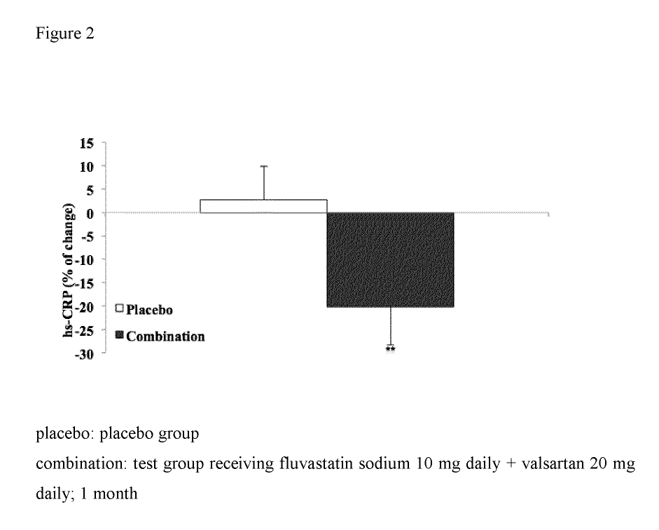

Significant decrease of levels of hsCRP was observed in individuals treated with the same pharmaceutical combination composition as in example 1, whereas no change was found in placebo group (FIG. 2).

### Example 3

### a) Subjects and Experimental Design

30 apparently healthy male individuals (54.9±3.1 years), who had a Framingham risk factor for a CHD risk (10 years) of 6.2, were included double blind, randomised study. They had no history of cardiovascular disease. The control group (n=15) received placebo, while the test group (n=15) received subtherapeutic daily dose of valsartan—20 mg daily and subtherapeutic daily dose of fluvastatin sodium—10 mg daily during a period of 1 month—30 days.

Application of the same pharmaceutical combination composition as in Example 1, same ultrasound measurement, same statistical analysis.

### b) Results

TABLE 4
Functional and morphological parameters before and after treatment

| | Before | After | |
|---|---|---|---|
| | treatment | treatment | Improvement |
| FMD (%) | 2.03 | 5.49 | +170.4% |
| PWV (m/s) | 6.11 | 5.60 | −9.1% |
| β-stiffness (U) | 7.90 | 7.00 | −12.9% |
| Arterial age according to age- | 50.0 | 41.0 | −9.0 |
| related normogram (years) | | | |

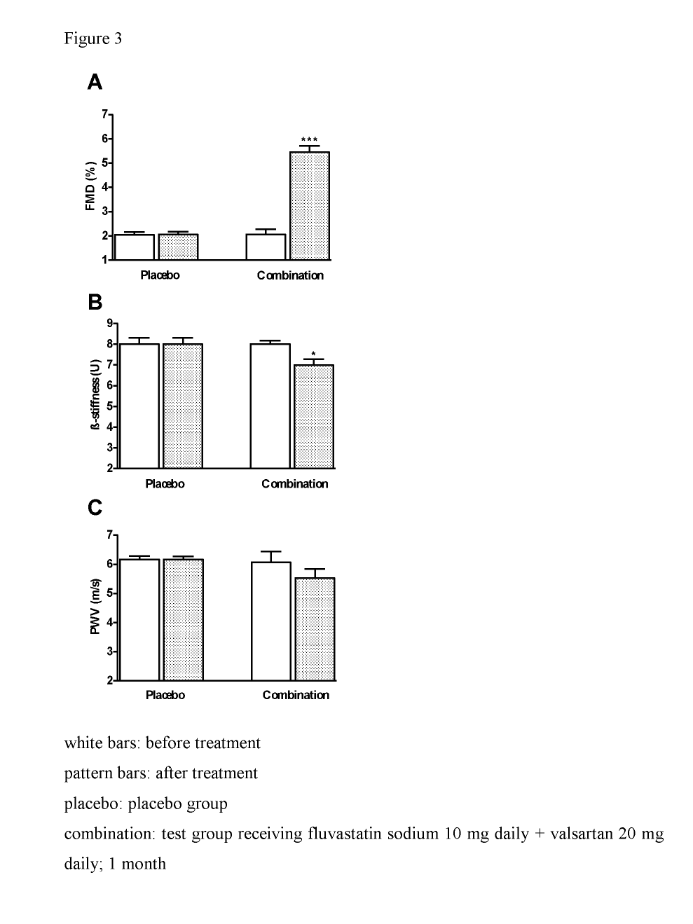

The results presented in Table 4 and FIG. 3 show that FMD increased by 170.4% (P<0.001; FIG. 3A), β-stiffness of the carotid artery decreased by 12.9% (P<0.05; FIG. 3B) and PWV decreased by 9.1% (FIG. 3C) after 1 month treatment period. No significant changes in described parameters in the placebo group throughout the study were observed.

### Example 4

Three apparently healthy individuals (43.3±1.5 years), who had a Framingham risk factor for a CHD risk (10 years) of 6.3, were treated with the same pharmaceutical combination composition as in example 1 to which coenzyme Q10 in a dosage of 50 mg two times daily was additionally added, all other parameters were the same as disclosed in Example 1.

TABLE 5
Functional and morphological parameters before and after treatment

| | Before | After | |
|---|---|---|---|
| | treatment | treatment | Improvement |
| FMD (%) | 2.11 | 7.83 | +271.1% |
| PWV (m/s) | 5.70 | 5.20 | −9.6% |
| β-stiffness (U) | 6.71 | 5.80 | −15.7% |

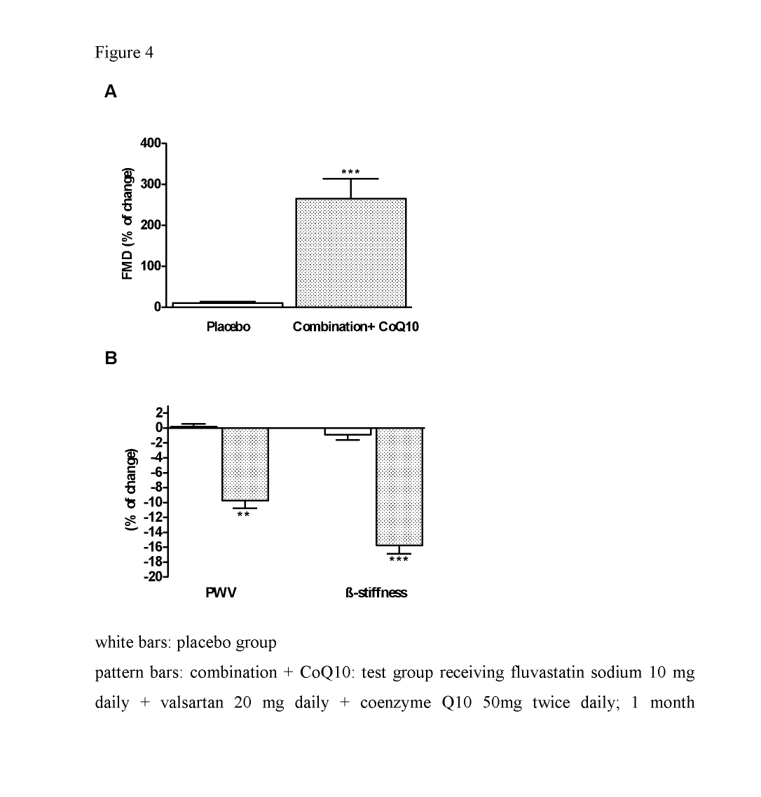

The substantial improvement was observed in each subject of the test group in all measured ultrasound parameters (FIG. 4).

### Example 5

Three apparently healthy individuals (43.7±1.8 years), who had a Framingham risk factor for a CHD risk (10 years) of 6.4, were treated with the same pharmaceutical combination composition as in example 1 to which coenzyme Q10 in a dosage of 50 mg two times daily and acetylsalicylic acid in a dosage of 100 mg once daily were additionally added, all other parameters were the same as in Example 1.

TABLE 6
Functional and morphological parameters before and after treatment

| | Before | After | |
|---|---|---|---|
| | treatment | treatment | Improvement |
| FMD (%) | 2.00 | 7.81 | +290.5% |
| PWV (m/s) | 5.58 | 5.04 | −10.7% |
| β-stiffness (U) | 6.40 | 5.51 | −16.2% |

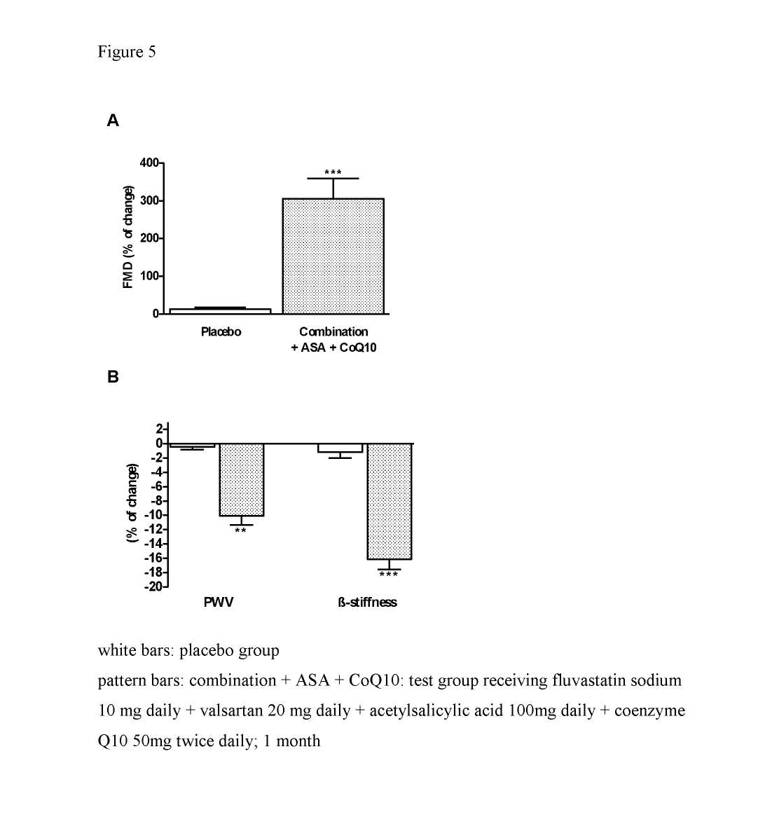

The substantial improvement was observed in each subject of the test group in all measured ultrasound parameters (FIG. 5).

### Example 6

Follow-up ultrasound measurements were repeated after 5, 7 and 8 months of therapy discontinuation in all participants of Example 1.

TABLE 7
Residual improvement expressed in % of effect achieved after 1 month of
treatment

| | Rest period | Rest period 7 | Rest period |
|---|---|---|---|
| | 5 months | months | 8 months |
| FMD (%) | 89.9% | 81.0% | 55.0% |
| PWV (m/s) | 75.3% | 70.0% | 26.4% |
| β-stiffness (U) | 72.3% | 68.0% | 23.2% |
| Arterial age according to age- | 36.2 (−8.8) | 37.3 (−7.7) | 41.1 (−3.9) |
| related normogram (years) | | | |

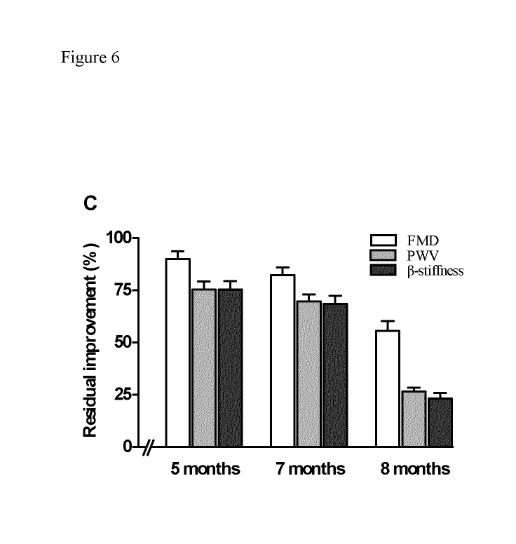

The substantial prolonged effect was clearly revealed (FIG. 6).

### Example 7

Follow-up ultrasound measurements were repeated after 5, 7 and 10 months of therapy discontinuation in all participants of Example 3.

TABLE 8
Residual improvement expressed in % of effect achieved after 1 month of
treatment

| | Rest period | Rest period 7 | Rest period |
|---|---|---|---|
| | 5 months | months | 10 months |
| FMD (%) | 92.2% | 85.0% | 24.3% |
| PWV (m/s) | 71.2% | 68.0% | 16.0% |
| β-stiffness (U) | 74.0% | 65.7% | 12.7% |
| Arterial age according to age- | 41.0 (−9.0) | 42.1 (−7.9) | 48.0 (−2.0) |
| related normogram (years) | | | |

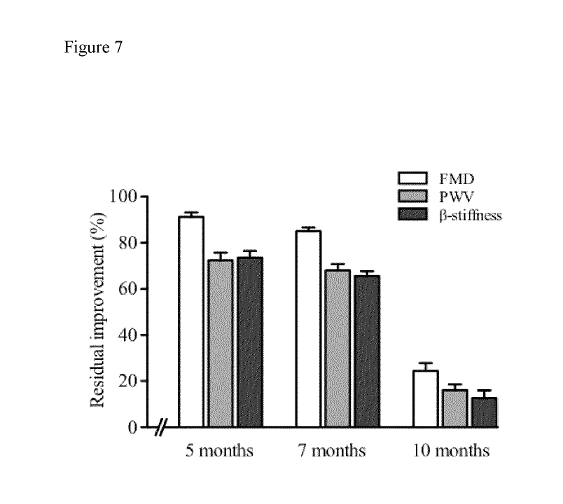

The important prolonged effect was clearly revealed (FIG. 7).

### Example 8

Follow-up ultrasound measurements were repeated after 7 months of treatment discontinuation in all participants of Example 1 (fluvastatin+valsartan group). In addition, twenty apparently healthy male individuals (43.5±1.6 years) for fluvastatin group (Table 9-1) (10 mg daily, 30 days, data from co-pending patent application), who had a Framingham risk factor for a CHD risk (10 years) of 6.7, and twenty apparently healthy male individuals (43.0±2.3 years) for valsartan group (Table 9-2) (20 mg daily, 30 days, data from co-pending patent application). who had a Framingham risk factor for a CHD risk (10 years) of 6.9, were recruited in double blind, randomized study wherein the inclusion criteria was the same as in Example 1.

TABLE 9-1
Functional and morphological parameters before and after treatment with
fluvastatin

| | Before | | |
|---|---|---|---|
| | treatment | After treatment | Improvement |
| FMD (%) | 2.31 | 4.39 | +91.0% |
| PWV (m/s) | 5.76 | 5.41 | −6.4% |
| β-stiffness (U) | 7.10 | 6.40 | −10.9% |
| Arterial age according to | 45.0 | 38.2 | −6.8 |
| age normogram (years) | | | |

The results presented in Table 9-1 shows that the FMD increased by 91.0%, β-stiffness of the carotid artery decreased by 10.9% and PWV decreased by 6.4% and 1 month treatment period with fluvastatin only.

TABLE 9-2
Functional and morphological parameters before and after treatment with
valsartan

| | Before treatment | After treatment | Improvement |
|---|---|---|---|
| FMD (%) | 2.11 | 5.40 | +156.0% |
| PWV (m/s) | 5.80 | 5.41 | −7.2% |
| β-stiffness (U) | 7.28 | 6.40 | −13.7% |
| Arterial age according | 45.0 | 38.0 | −7.0 |
| to age normogram | | | |
| (years) | | | |

The results presented in Table 9-2 show that FMD increased by 156.0%, β-stiffness of the carotid artery decreased by 13.7% and PWV decreased by 7.2% after 1 month treatment period with Valsartan only.

TABLE 9
Residual improvement after 7 months of rest period expressed in % of
effect achieved after 1 month of treatment

| | Fluvastatin | Valsartan | (fluvastatin + |
|---|---|---|---|
| | group | group | valsartan group) |
| FMD (%) | 33.0% | 30.4% | 81.0% |
| PWV (m/s) | 10.0% | 9.9% | 70.0% |
| β-stiffness (U) | 9.0% | 9.2% | 68.0% |
| Arterial age according to | 43.6 (−1.4) | 43.5 (−1.5) | 37.3 (−7.7) |
| age-related normogram | | | |
| (years) | | | |

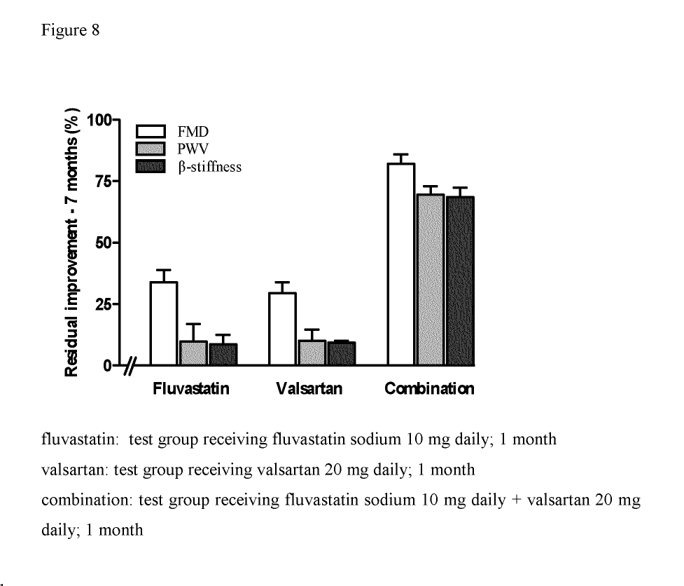

The results clearly show the high synergistic effect of the combination compared to drugs alone (FIG. 8). The results clearly show the synergistic effects of combination compared to actives alone. Synergistic effect is particularly substantial for parameters defining arterial stiffness (PWV and β-stiffness).

### Example 9

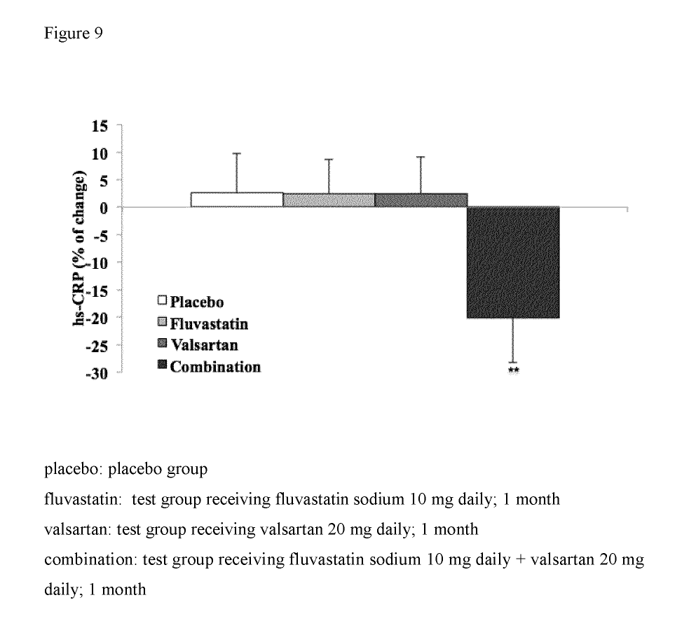

In the same participants as in Example 1 the levels of hsCRP were measured after 1 month treatment by standard method using Vitro 5.1 FS Chemical System (Ortho Clinical Diagnostics, Inc). The results clearly show the high synergistic effect of the combination compared to drugs alone (FIG. 9).

TABLE 10
Changes in hs-CRP values after treatment with fluvastatin, valsartan or
fluvastatin + valsartan

| | Fluvastatin | | (fluvastatin + valsartan) |
|---|---|---|---|
| | group* | Valsartan group* | group |
| Hs-CRP | +2.50% | +2.45% | −21.80% |

*calculated on data from co-pending patent application

### Example 10

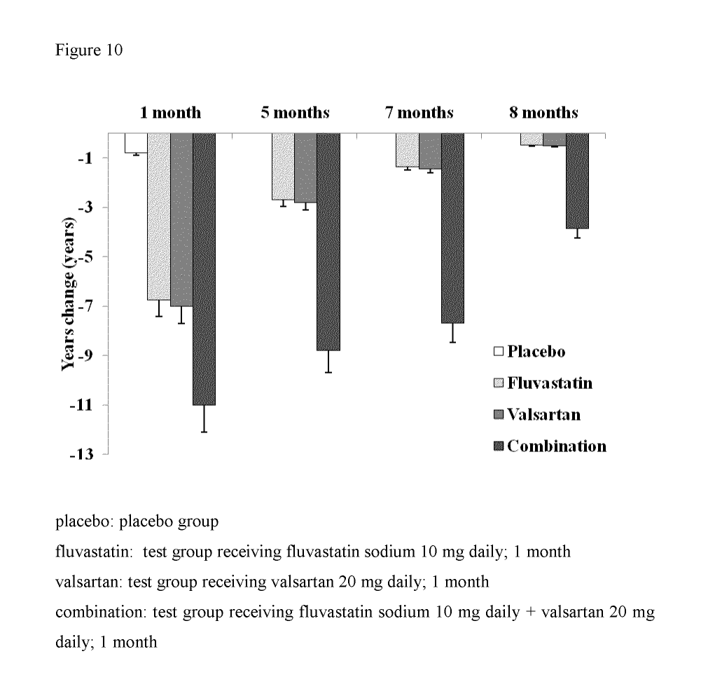

By using age-related normogram the “biological” arterial age in participants according to Example 1 after 1 month treatment and 5, 7 and 8 months of rest period and Examples from co-pending patent applications was calculated (according to the methods described in Jurasic M J et al. β-stiffness—setting age standards. Acta Clin Croat 2009; 48:253-8; and Carerj S. Normal vascular ageing evaluated by a new tool: e-tracking. Eur J Echocardiography 2006; suppl 1:S49.) The results clearly show the substantial synergistic effect achieved by the treatment according to Example 1 (FIG. 10)

### Example 11

Three apparently healthy elderly men (69.3±2.9 years), who had a Framingham risk factor for a CHD risk (10 years) of 8.2, three apparently healthy premenopausal women (42.3±4.2 years), who had a Framingham risk factor for a CHD risk (10 years) of 2.7 and three apparently healthy postmenopausal women (68.1±2.3 years), who had a Framingham risk factor for a CHD risk (10 years) of 6.7, were treated with the same pharmaceutical combination composition as in example 1, all other parameters were the same as in Example 1.

TABLE 11
Functional and morphological parameters before and after treatment

| | | After | |
|---|---|---|---|
| | Before treatment | treatment | Improvement |
| Elderly men | | | |
| FMD (%) | 1.60 | 4.10 | +156.2% |
| PWV (m/s) | 7.32 | 6.71 | −9.1% |
| β-stiffness (U) | 10.50 | 9.60 | −9.4% |
| Arterial age according to | 69.5 | 61.5 | −8.0 |
| age-related normogram | | | |
| (years) | | | |
| Premenopausal women | | | |
| FMD (%) | 3.05 | 5.39 | +76.7% |
| PWV (m/s) | 5.80 | 5.30 | −9.4% |
| β-stiffness (U) | 7.05 | 6.09 | −15.8% |
| Arterial age according to | 44.0 | 35.0 | −9.0 |
| age-related normogram | | | |
| (years) | | | |
| Postmenopausal women | | | |
| FMD (%) | 1.73 | 4.26 | +146.2% |
| PWV (m/s) | 7.00 | 6.30 | −11.1% |
| β-stiffness (U) | 9.62 | 8.67 | −10.9% |
| Arterial age according to | 68.5 | 56.5 | −12.0 |
| age-related normogram | | | |
| (years) | | | |

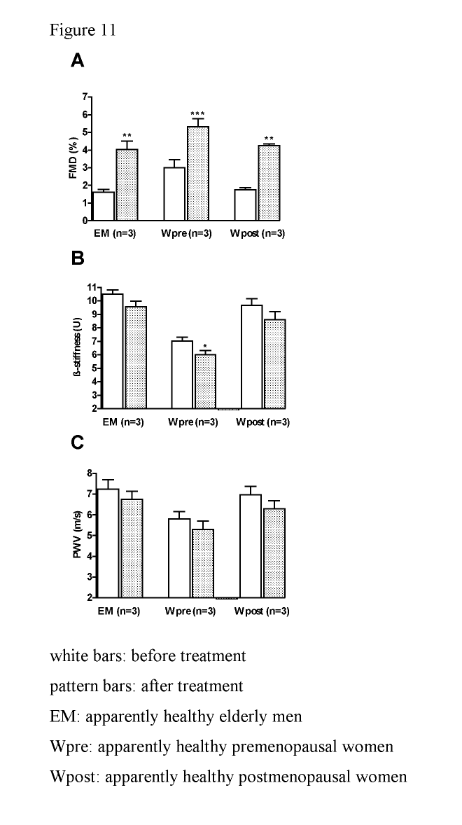

The substantial improvement was observed in each subject of the test group in all measured ultrasound parameters (FIG. 11).

### Example 12

### Animals and Experimental Design

In order to investigate whether a combination of low-dose statin (atorvastatin) and an ARB (losartan), separately not possessing protective cardiovascular pleiotropic effects, when combined express them, in vitro animal experiments on relaxation of thoracic aorta and ischemic-reperfusion injury of isolated rat heart were performed. The remarkable additive/synergistic effects were proved between low-doses of a statin (atorvastatin) and an ARB (losartan) resulting in substantial cardiovascular protection. Adult male Wistar rats (n=40) weighing 260-300 g were obtained from the Faculty of Medicine, Ljubljana. They were bred under constant housing conditions and fed with standard rat chow in the form of pellets (Altromin No. 1320, Lage, Germany); a regular circadian cycle, lasting for 12 h was maintained. Five to seven animals were kept in each cage at a controlled environmental temperature. All animal procedures were conducted in accordance with the guidelines set by the Veterinary Administration of the Republic of Slovenia (permit No. 34401-23/2009/3).

The Wistar rats were anaesthetized and their thoracic aortas and hearts were isolated. Relaxation of aortic rings, coronary flow rate and the extent of myocardial ischaemic-reperfusion injury were measured. Low concentrations of atorvastatin or losartan (e.g. 0.1 and 1 μM, respectively) added to a perfusion medium were tested. Further, combinations of atorvastatin and losartan added to a perfusion medium were tested.

### Data Acquisition and Analysis

Vascular responses and data for all parameters measured in isolated hearts were recorded and processed on a Dewetron acquisition system (Dewetron, Graz, Austria) after analogue-digital conversion (National Instruments, NI PCI-6013, Austin, USA) on the hard disk of a personal computer by Dewesoft 6.0 software (Dewetron, Trbovlje, Slovenia).

Statistical analysis was performed using GraphPad Prism 5.0 (GraphPad Software, San Diego Calif., USA). Values were expressed as means±SEM for n observations, where n represents the number of animals/hearts and m represents the number of aortic rings used in each of the studied groups. In isolated heart experiments and comparison of endothelium-dependent vasorelaxation, one-way analysis of variance (ANOVA) with Bonferroni's post-test was used to compare the studied groups. Thoracic aorta relaxations were compared by two-way analysis of variance (ANOVA) with Bonferroni's post-test used to compare the studied groups. A value of P<0.05 was considered significant.

### Relaxation of Aortic Rings

Thoracic aorta endothelium-dependent relaxation was measured with acetylcholine in aortic rings precontracted with phenylephrine after incubation in different concentrations of the drugs.

After isolation, aortic rings were immediately mounted in standard organ baths filled with K-H (Krebs-Henseleit) solution. After mounting, equilibration of rings at 20 nm resting tension was performed, periodically adjusting it to the desired level and changing the K-H solution every 10 minutes. The equilibration period lasted for 60 min. Later, rings were contracted with 60 mM KCl until reproducible contractile responses were obtained. Then the rings were precontracted with 1 μmol/L phenylephrine (Phe) before addition of 1 μmol/L acetylcholine (ACh) to check the presence of a functional endothelium.

Following washout, aortic rings were incubated for 30 minutes in atorvastatin, losartan and their combination or in K-H solution (control group). After another washout the endothelium dependent relaxation was remeasured, and rings were precontracted with 1 μM phenylephrine before addition of 1 μM acetylcholine (Ach) to measure the endothelium dependent vasorelaxation.

The following effective concentrations were found for endothelium dependent relaxation:

TABLE 12
Endothelium dependent relaxation after treatment with atorvastatin,
losartan and their low-dose combination (atorvastatin + losartan)

| | atorvastatin | losartan | atorvastatin + losartan |
|---|---|---|---|
| Effective concentration | 1 μM | 10 μM | 0.1 μM + 1 μM |
| Endothelium | 21.1% | 17.0% | 26.3% |
| dependent | | | |
| relaxation compared to | (P < 0.001) | (P < 0.01) | (P < 0.001) |
| controls | | | |

*The drugs used separately at the same doses as used in the combination, had no influence

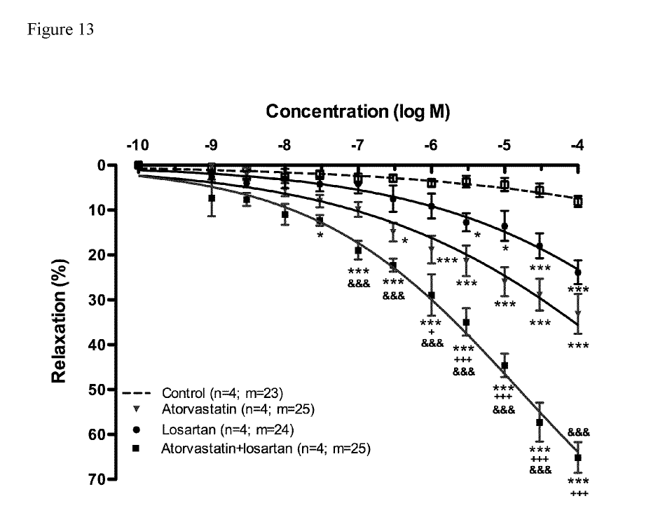

FIG. 13 shows the effects of cumulative addition of atorvastatin, losartan, and their combination on isolated rat aortic rings precontracted with 1 μM phenylephrine.

### Coronary Flow Rate:

In the control group, hearts were perfused with oxygenated K-H solution during the first 30 min (perfusion phase), followed by 40 min of global zero flow ischaemia with complete flow cessation of K-H solution to the isolated heart. After that, hearts were perfused with oxygenated K-H solution for 50 min (reperfusion period).

In the test groups, hearts were perfused with oxygenated K-H for the first 20 min only, followed by 10 min perfusion of K-H solution with the added drugs atorvastatin or losartan in different concentrations of 0.01 μM, 0.1 μM or 1 μM, or a low-dose combination of atorvastatin (0.01 μM) and losartan (0.1 μM). Then 40 min of global zero flow ischaemia was applied, followed by 50 min of reperfusion with K-H solution with added drugs.

In order to measure the coronary flow rate, the coronary effluent was collected in a calibrated test tube at various time intervals during the perfusion and reperfusion periods, recording the effluent volume.

The following effective concentrations were found for an increase of the coronary flow:

TABLE 13
Increase in coronary flow after treatment with atorvastatin, losartan and
their low-dose combination (atorvastatin + losartan)

| | | | atorvastatin + |
|---|---|---|---|
| | atorvastatin | losartan | losartan |
| Effective concentration | 0.1 μM | 1 μM | 0.01 μM + 0.1 μM* |
| Increase in coronary | 52.9% | 67.3% | 68.9% |
| flow compared to | (P < 0.05) | (P < 0.001) | (P < 0.001) |
| controls | | | |

*The drugs used separately at the same doses as used in the combination had no influence

### Extent of Myocardial Ischaemic-Reperfusion Injury

The reduction of myocardial ischaemic-reperfusion injury is measured by the LDH (Lactate dehydrogenase) release rate. The collected coronary effluents during the perfusion and reperfusion period were used for the analysis of the LDH release activity. LDH release rate activity was determined by the modified Wroblewski-LaDue method (Wroblewski F. and Ladue J S., Proc. Soc. Exp. Biol. (1955), 90(1), 210-213).

LDH is a cytosolic intracellular enzyme released due to cell membrane damage. From analysis of coronary effluent, LDH release rates were obtained in order to assess the degree of cardiac tissue injury. At the beginning of the experiments, LDH release rates were increased due to the preparation procedure and decreased by the 30th minute, when constant and approximately equal values were reached in all heart groups.

The values of LDH release rates steadily rose in the control group during reperfusion, reaching maximum in the 110^{th} and 120^{th} minute of the experiments. Instead, the LDH release rates decreased in the test groups at the end of the reperfusion period, i.e. in the 110^{th} and 120^{th} minute.

The following effective concentrations were found for a decrease of the LDH release rates:

TABLE 14
Decrease of LDH release rates after treatment with atorvastatin, losartan or
their low-dose concentration (atorvastatin + losartan)

| | | | atorvastatin + |
|---|---|---|---|
| | Atorvastatin | losartan | losartan |
| Effective concentration | 0.1 μM | 1 μM | 0.01 μM + 0.1 μM* |
| Decrease of the LDH | 151.1% | 58.6% | 184.2% |
| release rates compared | (P < 0.001) | (P < 0.01) | (P < 0.001) |
| to controls | | | |

*The drugs used separately at the same doses as used in the combination had no influence

### Example 13

### Subjects and Experimental Design

Sixty Wistar rats of both sexes weighing 230-280 g, 20-24 weeks of age, were obtained from the Faculty of Medicine, Ljubljana. They were bred under constant housing conditions and fed with standard rat chow in the form of pellets (Altromin No. 1320, Lage, Germany). A regular 12 h circadian cycle was maintained and between 5 and 7 animals were kept in each cage. The temperature of the experimental environment was 23-25° C. All animal procedures were conducted in accordance with the guidelines set by the Veterinary Administration of the Republic of Slovenia (permit No. 34401-23/2009/3).

The rats were randomly assigned to one of four experimental groups as follows: 1) rats that received tap water (controls); 2) rats that received atorvastatin (2 mg/kg/day; p.o.); 3) rats that received losartan (5 mg/kg/day; p.o.), and 4) rats that received a combination of atorvastatin and losartan (2 mg/kg/day p.o. and 5 mg/kg/day p.o., respectively). After 4-8 weeks of therapy the animals were sacrificed, blood samples were collected and heart and thoracic aorta were isolated. Each group was composed of the same number of male and female rats.

The coronary flow rate and the endothelium-dependent relaxation were measured.

### Data Acquisition and Analysis

Vascular responses were processed and recorded on a Dewetron acquisition system (Dewetron, Graz, Austria) after analogue-digital conversion (National Instruments, NI PCI-6013, Austin, USA) on the hard disk of a personal computer by Dewesoft 6.0 software (Dewetron, Trbovlje, Slovenia).

Statistical analysis was performed using GraphPad Prism 5.0 (GraphPad Software, San Diego Calif., USA). Values were expressed as means±S.E.M for n observations, where n represents the number of animals and m represents the number of aortic rings used in each of the studied groups. The relative relaxation responses of arterial rings were expressed as the percentage of phenylephrine precontracted aortic rings. Isolated thoracic aorta results were fitted and plotted using sigmoidal concentration-response curves. Two-way analysis of variance (ANOVA) with Bonferroni's post-test was used to make inter-group comparisons. A value of P<0.05 was considered significant.

### Relaxation of Aortic Rings

Thoracic aorta endothelium-dependent relaxation was measured with acetylcholine in aortic rings precontracted with phenylephrine after incubation in different concentrations of the drugs.

After isolation, aortic rings were immediately mounted in standard organ baths filled with K-H (Krebs-Henseleit) solution. After mounting, rings were allowed to equilibrate at 20 mN resting tension (found to be optimal in previous experiments) for 60 min. During this period, the tension was periodically adjusted to the desired level and the K-H solution was changed every 10 minutes. After the equilibration period, the rings were contracted three times with 60 mmol/L KCl to achieve stable contractions. After rinsing, the relaxation of precontracted rings with the al adrenergic agonist phenylephrine (1 mmol/L) was examined by adding cumulative concentrations of the endothelium dependent dilator acetylcholine (ACh) (0.1 nmol/L-1 mmol/L).

In control animals the endothelium-dependent relaxation of the thoracic aorta was constant. However, in rats treated with drugs the percentage of endothelium-dependent relaxation was increased in comparison to the control group.

The following increases of endothelium dependent relaxation were measured in rats treated for 6 weeks with atorvastatin, losartan, and atorvastatin+losartan:

TABLE 15
Increase of endothelium dependent relaxation after 6 weeks treatment with
atorvastatin, losartan or their combination (atorvastatin + losartan)

| | Atorvastatin | losartan | atorvastatin + losartan |
|---|---|---|---|
| Daily dose | 5 mg/kg | 2 mg/kg | 5 mg/kg + 2 mg/kg |
| Increase of endothelium | 6.9% | 7.3% | 21.7% |
| dependent relaxation | NS | NS | (P < 0.05) |
| compared to controls | | | |

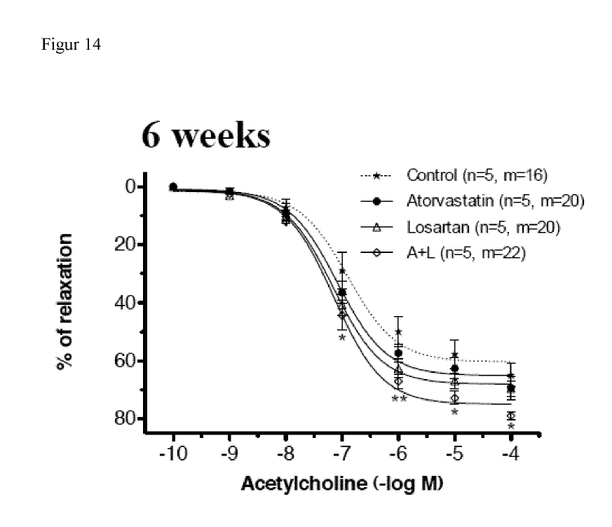

FIG. 14 shows the effects of six weeks treatment with atorvastatin, losartan, and their combination on acetylcholine-induced endothelium-dependent vasorelaxation.

### Coronary Flow Rate:

In order to measure the coronary flow rate, the coronary effluent was collected in a calibrated test tube at various time intervals during the perfusion and reperfusion periods, recording the effluent volume.

The following increases of the coronary flow were measured in rats treated for 6 weeks with atorvastatin, losartan, and atorvastatin+losartan:

TABLE 16
Increase in coronary flow after 6 weeks treatment with atorvastatin,
losartan or their combination (atorvastatin + losartan)

| | atorvastatin | losartan | atorvastatin + losartan |
|---|---|---|---|
| Daily dose | 5 mg/kg | 2 mg/kg | 5 mg/kg + 2 mg/kg |
| Increase in coronary | 86.9% | 89.6% | 149.2% |
| flow compared to | (P < 0.001) | (P < 0.001) | (P < 0.001) |
| controls | | | |

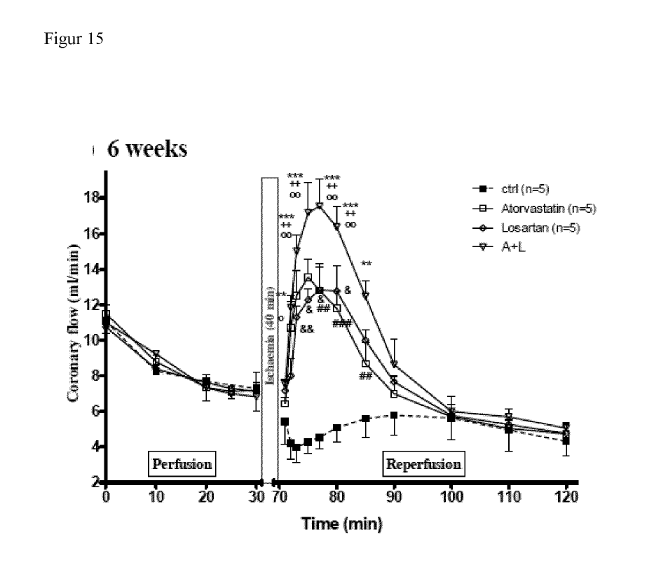

FIG. 15 shows the effects of six weeks treatment with atorvastatin, losartan, and their combination on the coronary flow values in experiments with 40-minute ischemia, followed by reperfusion.

### Example 14

Results of repetition of treatment period after rest period of 12 months (ten participants, fluvastatin 10 mg daily and valsartan 20 mg daily for 1 month—30 days).

TABLE 17
Improvement on FMD, PWV and β-stiffness achieved after 1 month of
treatment (1. intervention) and a further 1 month treatment
(2. intervention) after a rest period of 12 months

| | 1. intervention | | 2. intervention (repetition) | |
|---|---|---|---|---|
| | before | After | before | after |
| FMD (%) | 4.1 ± 0.4 | 6.7 ± 0.3 | 4.4 ± 0.4 | 6.9 ± 0.5 |
| β-stiffness (U) | 7.6 ± 0.4 | 6.4 ± 0.4 | 7.7 ± 0.5 | 6.5 ± 0.4 |
| PWV (m/s) | 5.9 ± 0.3 | 5.3 ± 0.3 | 5.8 ± 0.2 | 5.2 ± 0.3 |

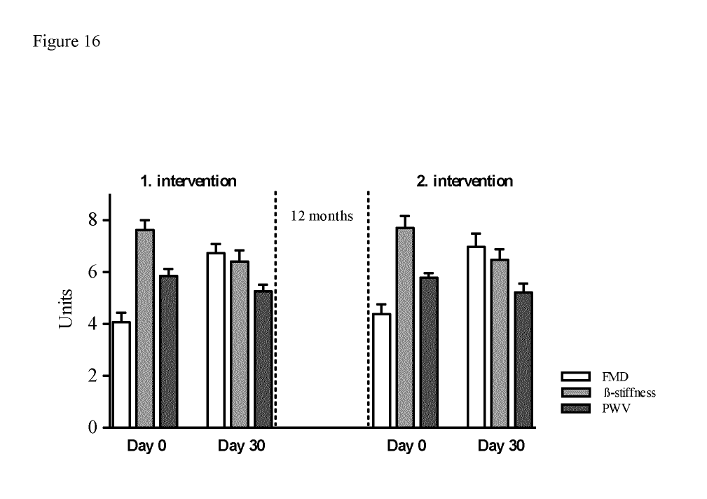

The results of the FMD, PWV and β-stiffness measurements after the 1. intervention cycle and the 2. intervention cycle are shown in FIG. 16. Evidently, the beneficial effects can be repeated.

1. A pharmaceutical composition comprising
at least one renin-angiotensin-aldosterone system inhibitor in a subtherapeutic daily dose, and
at least one HMG-CoA reductase inhibitor in a subtherapeutic daily dose,
for use in the prevention, reduction or reversal of arterial ageing in apparently healthy subjects.

2. A pharmaceutical composition comprising
at least one renin-angiotensin-aldosterone system inhibitor in a subtherapeutic daily dose, and
at least one HMG-CoA reductase inhibitor in a subtherapeutic daily dose,
for use in decreasing the occurrence of cardiovascular disorders in apparently healthy subjects.

3-115. (canceled)
